(19) United States
(12) Patent Application Publication
(10) Pub. No.: US 2026/0094670 A1
(43) Pub. Date: Apr. 2, 2026

(54) METHOD FOR OBTAINING PREDICTION RESULT OF NUCLEIC ACID AMPLIFICATION REACTION, METHOD FOR OBTAINING MODEL PROVIDING PREDICTION RESULT OF NUCLEIC ACID AMPLIFICATION REACTION AND COMPUTER DEVICE FOR PERFORMING SAME

(71) Applicant: Seegene, Inc., Seoul (KR)

(72) Inventors: Hyeon Be KIM, Seoul (KR); Han Bit LEE, Seoul (KR); Jeong Woo KIM, Seoul (KR); Iljae MIN, Seoul (KR)

(21) Appl. No.: 19/411,877

(22) Filed: Dec. 8, 2025

Related U.S. Application Data
(63) Continuation of application No. PCT/KR2024/007766, filed on Jun. 7, 2024.

(30) Foreign Application Priority Data
Jun. 9, 2023 (KR) 10-2023-0074357

(51) Int. Cl. G16B 25/20; G16B 40/10; G16B 40/20

(52) U.S. Cl. CPC G16B 25/20; G16B 40/10; G16B 40/20

(57) ABSTRACT

Proposed is a method for obtaining a prediction result of a nucleic acid amplification reaction affected by an nth-order structure, which is performed by a computing device. The method may include accessing a prediction model learned using a plurality of training data Each training data may include a first analysis data for an nth-order structure in a nucleic acid sequence and an amplification reaction result for the nucleic acid sequence, where n is an integer not less than 2. The method may also include obtaining an input data comprising a second analysis data for an nth-order structure in a target nucleic acid sequence. The method may further include providing the input data to the prediction model, and obtaining a prediction result of an amplification reaction for the target nucleic acid sequence from the prediction model.

### CROSS REFERENCE TO RELATED APPLICATIONS

This is a continuation application of International Patent Application No. PCT/KR2024/007766 filed on Jun. 7, 2024, which claims priority to Korean patent application No. 10-2023-0074357 filed on Jun. 9, 2023, contents of each of which are incorporated herein by reference in their entireties.

### BACKGROUND

### Technical Field

The disclosure relates to a method for obtaining a prediction result of a nucleic acid amplification reaction, a method for obtaining a model providing predication result of a nucleic acid amplification reaction, and a computer device for performing the same

### Description of Related Technology

Molecular diagnosis is a rapidly growing field in the in vitro diagnostic market for early diagnosis of diseases. Among them, nucleic acid-based methods are useful for diagnosing genetic factors caused by infection by virus or bacteria based on their high specificity and sensitivity.

### SUMMARY

One aspect is predicting to what extent a nucleic acid amplification reaction for a nucleic acid sequence is affected by the formation of a secondary structure in the corresponding nucleic acid sequence.

Another aspect is allowing a target nucleic acid to be accurately detected using an oligonucleotide in consideration of a correlation between the secondary structure and a nucleic acid amplification reaction during a design process of the oligonucleotide.

The aspects are not limited to those described herein, and other aspects that are not mentioned may be clearly understood by those of ordinary skill in the art to which the present disclosure belongs from the following description.

Another aspect is a method for obtaining a prediction result of a nucleic acid amplification reaction affected by an nth-order structure, which is performed by a computing device, the method comprising accessing a prediction model learned using a plurality of training data, each training data comprises a first analysis data for an nth-order structure in a nucleic acid sequence and an amplification reaction result for the nucleic acid sequence; wherein n is an integer not less than 2; obtaining an input data comprising a second analysis data for an nth-order structure in a target nucleic acid sequence; providing the input data to the prediction model; and obtaining a prediction result of an amplification reaction for the target nucleic acid sequence from the prediction model.

In an embodiment, wherein n is 2, and the nth-order structure comprises at least one selected from the group consisting of a hairpin loop, an internal loop, a bulge loop, multi-loops, a G-quadruplex, and a combination thereof.

In an embodiment, wherein the first analytical data and the second analytical data each comprises a thermodynamic data for a formation of an nth-order structure in the corresponding nucleic acid sequence.

In an embodiment, wherein the thermodynamic data for the formation of the nth-order structure is a thermodynamic data for a formation of an arbitrary nth-order structure, a thermodynamic data for a formation of a specific nth-order structure, or a thermodynamic data for a formation of each of a plurality of nth-order structures.

In an embodiment, wherein the thermodynamic data is indicated as a change in a thermodynamic free energy.

In an embodiment, wherein the thermodynamic data comprises at least one selected from the group consisting of: (a) a thermodynamic data for an nth-order structure present in a first block unit; wherein the first block unit is defined as a predetermined range based on a region in which an oligonucleotide is bound to the corresponding nucleic acid sequence during an annealing step of the amplification reaction; (b) a thermodynamic data for an nth-order structure present in a second block unit; wherein the second block unit is defined as a range of a region to be extended by an oligonucleotide bound to the corresponding nucleic acid sequence during an extension step of the amplification reaction, and (c) a thermodynamic data for an nth-order structure present in a third block unit; wherein the third block unit is defined as a sequence comprising (i) the second block and (ii) an additional sequence at a 5′ end and a 3′ end of the second block.

In an embodiment, wherein the thermodynamic data is obtained based at least in part on the corresponding nucleic acid sequence and a reaction condition used in the amplification reaction for the corresponding nucleic acid sequence, and wherein the reaction condition comprises a condition for a reaction medium and temperature used in the corresponding amplification reaction.

In an embodiment, wherein the each training data further comprises at least one selected from the group consisting of: (a) at least one of the nucleic acid sequence, an amplicon sequence obtained from the nucleic acid sequence, and an oligonucleotide sequence bound to the nucleic acid sequence; (b) a melting temperature (Tm) of at least one of the nucleic acid sequence, the amplicon sequence, and the oligonucleotide sequence; (c) a length of at least one of the nucleic acid sequence, the amplicon sequence, and the oligonucleotide sequence; (d) a type of the nucleic acid sequence; and (e) a GC content of at least one of the nucleic acid sequence, the amplicon sequence, and the oligonucleotide sequence.

In an embodiment, wherein the amplification reaction result comprises at least one selected from the group consisting of (a) an amplification inhibition level representing a level at which the amplification reaction is inhibited by the nth-order structure, and (b) an amplification inhibition or an amplification non-inhibition representing whether the amplification inhibition level satisfies a predetermined criterion.

In an embodiment, wherein the amplification inhibition level is calculated using a cycle value corresponding to an amplification point in a dataset comprising a signal value for each cycle for the amplification reaction.

In an embodiment, wherein the cycle value corresponding to the amplification point comprises (i) a cycle value in which a primary or a secondary derivative result for a curve connecting the signal value for each cycle is maximum or the minimum and/or (ii) a specific cycle value in which a signal value in the dataset reaches a preset threshold value.

In an embodiment, wherein the amplification inhibition level is calculated using a difference between the amplification points determined from two or more datasets obtained from two or more amplification reactions for the nucleic acid sequence.

In an embodiment, wherein the predetermined criterion is determined based at least in part on a value of n and/or a type of the nth-order structure in the nucleic acid sequence.

In an embodiment, wherein the type of the nth-order structure comprises: at least one selected from the group consisting of a hairpin loop, an internal loop, a bulge loop, multi-loops, a G-quadruplex, and a combination thereof, when n is 2; and at least one selected from the group consisting of a pseudoknot, a kissing hairpin, a hairpin-bulge contact, and a combination thereof, when n is 3.

In an embodiment, wherein the prediction model comprises at least one selected from the group consisting of a machine learning-based Ridge linear regression model, a random forest regression model, a logistic regression-based classification model, and a random forest classification model.

In an embodiment, wherein the prediction result comprises at least one selected from the group consisting of (a) an amplification inhibition level representing a level at which an amplification reaction is inhibited by the nth-order structure, (b) an amplification inhibition or an amplification non-inhibition representing whether the amplification inhibition level satisfies a predetermined criterion, and (c) a probability value for the amplification inhibition or the amplification non-inhibition.

In an embodiment, wherein the input data comprises a plurality of feature, and the method further comprises, after the obtaining of the prediction result, providing a contribution level representing a level to which each of the plurality of feature contributed to the prediction result.

In an embodiment, wherein the method further comprises determining a designable region of an oligonucleotide based at least in part on the prediction result.

In an embodiment, wherein the amplification reaction is a Polymerase chain reaction (PCR).

Another aspect is a computer device that comprises a memory configured to store at least one instruction; and a processor, wherein the at least one instruction is executed by the processor to: (a) access a prediction model learned using a plurality of training data, each training data comprises a first analysis data for an nth-order structure in a nucleic acid sequence and an amplification reaction result for the nucleic acid sequence; wherein n is an integer not less than 2; (b) obtain an input data comprising a second analysis data for an nth-order structure in a target nucleic acid sequence; (c) provide the input data to the prediction model; and (d) obtain a prediction result of an amplification reaction for the target nucleic acid sequence from the prediction model.

Another aspect is a computer-readable recording medium that stores a computer program, wherein the computer program comprising an instruction that, when executed by one or more processors, enables the one or more processors to perform a method for obtaining a prediction result of a nucleic acid amplification reaction affected by an nth-order structure, wherein the method comprises: (a) accessing a prediction model learned using a plurality of training data; each training data comprises a first analysis data for an nth-order structure in a nucleic acid sequence and an amplification reaction result for the nucleic acid sequence; wherein n is an integer not less than 2; (b) obtaining an input data comprising a second analysis data for an nth-order structure in a target nucleic acid sequence; providing the input data to the prediction model; and obtaining a prediction result of an amplification reaction for the target nucleic acid sequence from the prediction model.

Another aspect is a computer program stored in a computer-readable recording medium that comprises an instruction that, when executed by one or more processors, enables the one or more processors to perform a method for obtaining a prediction result of a nucleic acid amplification reaction affected by an nth-order structure, wherein the method comprises: (a) accessing a prediction model learned using a plurality of training data; each training data comprises a first analysis data for an nth-order structure in a nucleic acid sequence and an amplification reaction result for the nucleic acid sequence; wherein n is an integer not less than 2; (b) obtaining an input data comprising a second analysis data for an nth-order structure in a target nucleic acid sequence; (c) providing the input data to the prediction model; and (d) obtaining a prediction result of an amplification reaction for the target nucleic acid sequence from the prediction model.

Another aspect is a method for obtaining a prediction model for providing a prediction result of a nucleic acid amplification reaction affected by an nth-order structure, which is performed by a computing device, the method comprising: (1) obtaining the following data group: (a) a plurality of first data groups; each first data group comprising a predetermined nucleic acid sequence and a reaction condition used for an amplification reaction for the nucleic acid sequence; wherein the nucleic acid sequence and/or the reaction condition of the plurality of first data groups are at least partially different; and (b) a plurality of second data groups determined from the plurality of first data groups; each second data group comprising at least one selected from the group consisting of: (i) an analysis data for an nth-order structure in the nucleic acid sequence; wherein n is an integer not less than 2; wherein the analysis data is determined using the nucleic acid sequence and the reaction condition in each first data group; (ii) a numerical data for an amplification inhibition level; wherein the amplification inhibition level represents a level at which the amplification reaction is inhibited by the nth-order structure in an amplification region; wherein the amplification region is determined from two or more datasets obtained from two or more amplification reactions to the nucleic acid sequence in each first data group, performed under the reaction condition; and (iii) a label data for an amplification inhibition or an amplification non-inhibition representing whether the amplification inhibition level satisfies a predetermined criterion; and (2) obtaining a prediction model learned to predict an amplification reaction result of the nucleic acid sequence, using at least one of the plurality of first data groups and the plurality of second data groups.

Another aspect is a method for determining a designable region of an oligonucleotide, which is performed by a computer device, the method comprising: (a) accessing a prediction model learned using a plurality of training data; each training data comprises a first analysis data for an nth-order structure in a nucleic acid sequence and an amplification reaction result for the nucleic acid sequence; wherein n is an integer not less than 2; (b) obtaining an input data comprising a second analysis data for an nth-order structure in a target nucleic acid sequence; (c) providing the input data to the prediction model; (d) obtaining a prediction result of an amplification reaction for the target nucleic acid sequence from the prediction model; and (e) determining a designable region of an oligonucleotide based at least in part on the prediction result.

In an embodiment, wherein the determining of the designable region comprises: (i) obtaining one or more candidate regions for the designable region; wherein the candidate region consists of a part of a reference nucleic acid sequence; wherein the reference nucleic acid sequence comprises a unique genome sequence corresponding to an organism; and (ii) determining a region to be excluded from the one or more candidate regions based on the prediction result.

According to an embodiment of the present disclosure, based on the analysis data for the nth-order structure present in the nucleic acid sequence, it is possible to predict the amplification reaction result as to whether the nth-order structure in the corresponding nucleic acid sequence affects the amplification reaction. For example, it is possible to predict the level or possibility that a nucleic acid amplification reaction for a nucleic acid sequence will be inhibited by the secondary structure present in the nucleic acid sequence. In addition, as such prediction is performed by applying the reaction conditions used for the amplification reaction, prediction accuracy may be further improved.

In addition, by using a model trained with datasets obtained under a predetermined nucleic acid sequence and reaction conditions, characteristics that are difficult to be analyzed by a human are reflected and used for prediction of an amplification reaction result affected by an nth-order structure, and thus, it is effective in terms of prediction performance. In addition, as a designable region of the oligonucleotide is determined based on the prediction result, the design of the oligonucleotide may be made for a region which is not affected by the formation of the nth-order structure. Accordingly, the accuracy of detection of the target nucleic acid using the oligonucleotide may be further improved.

It should be understood that the effects of the present disclosure are not limited to the above-described effects, and include all effects that can be deduced from the detailed description of the present disclosure, or the configuration of the invention described in Claims.

### BRIEF DESCRIPTION OF THE DRAWINGS

FIG. 1 schematically illustrates a block diagram of a computer device according to an embodiment.

FIG. 2 is a diagram illustrating modularized software implemented by the computer device shown in FIG. 1.

FIGS. 3A-3D show a diagram illustrating types of secondary structures of nucleic acid sequences according to an embodiment.

FIGS. 4A-4C show a diagram illustrating types of tertiary structures of nucleic acid sequences according to an embodiment.

FIG. 5 is a diagram for exemplarily describing thermodynamic data for an nth-order structure present in a nucleic acid sequence according to an embodiment.

FIG. 6 is a diagram for exemplarily describing the first to the third thermodynamic data for an nth-order structure in which a process of an amplification reaction for a nucleic acid sequence is considered, according to an embodiment.

FIGS. 7A-7D show a diagram illustrating an amplification point, an amplification region, and a specific cycle value used to calculate an amplification inhibition level according to an embodiment.

FIG. 8 is a diagram illustrating an amplification inhibition level according to an embodiment.

FIG. 9 exemplarily illustrates a conceptual diagram of a learning process of a prediction model according to an embodiment.

FIG. 10 is a conceptual diagram for describing a structure and an operation of a random forest-based prediction model according to a first embodiment.

FIG. 11 illustrates an example flowchart for obtaining a prediction model by the computer device according to an embodiment.

FIG. 12 illustrates an example flowchart for obtaining the prediction model by the computer device according to another embodiment.

FIG. 13 illustrates a conceptual diagram of a process in which a prediction model outputs a prediction result of a nucleic acid amplification reaction affected by an nth-order structure, according to an embodiment.

FIGS. 14A and 14B show a diagram illustrating an operation of providing a contribution level by the computer device according to an embodiment.

FIG. 15 illustrates an exemplary flowchart for obtaining a prediction result of a nucleic acid amplification reaction affected by an nth-order structure, by the computer device according to an embodiment.

FIG. 16 illustrates an exemplary flowchart for determining a designable region of an oligonucleotide according by the computer device to an embodiment.

### DETAILED DESCRIPTION

In most diagnostic methods using nucleic acids, a nucleic acid amplification reaction is used to amplify a target nucleic acid (e.g., a viral or bacterial nucleic acid). As a representative example, in a Polymerase chain reaction (PCR) among nucleic acid amplification reactions, a repeated cycle process of denaturation of double-stranded DNA, annealing of oligonucleotide primers to a DNA template, and primer extension by DNA polymerase is performed (Mullis et al., U.S. Pat. Nos. 4,683,195, 4,683,202, and 4,800,159; Saiki et al., Science 230: 1350-1354 (1985)).

As other methods for amplifying nucleic acids, various methods such as LCR (Ligase Chain Reaction), SDA (Strand Displacement Amplification), NASBA (Nucleic Acid Sequence-Based Amplification), TMA (Transcription Mediated Amplification), RPA (Recombinase Polymerase Amplification), LAMP (Loop-mediated isothermal amplification), and RCA (Rolling-Circle Amplification) have been proposed.

Among PCR-based techniques, real-time PCR is a technique for detecting target nucleic acids in a sample in real-time. In order to detect a specific target nucleic acid, a signal generation means that emits a detectable fluorescent signal in proportion to the amount of the target nucleic acid during the PCR reaction is used. A fluorescence signal proportional to the amount of the target nucleic acid is detected at each measurement point (cycle) through real-time PCR, and a dataset including each measurement point and a signal value at the measurement point is obtained. Additionally, an amplification curve or an amplification profile curve indicating the intensity of the detected fluorescence signal compared to the measurement point is obtained from the dataset.

In order to effectively detect a target nucleic acid, an oligonucleotide (probe and/or primer) used to detect the target nucleic acid should have proper specificity and sensitivity, and should be suitable for a specific detection method and should meet the conditions set by an analyst. Therefore, the design of oligonucleotides suitable for the purpose of analysis is very important.

Many techniques have been proposed in relation to the design of oligonucleotides (Nielsen H B et al., Nucleic Acids Res 31:3491-3496 (2003); Rouillard J M et al., Nucleic Acids Res 31:3057-3062 (2003); Wang X, et al., Bioinformatics 19:796-802 (2003); and Hu G, et al., BMC Bioinformatics 8:350 (2007)). In particular, in the case of a target nucleic acid molecule (particularly, a genomic sequence of an RNA virus) having sequence variability (genetic diversity), a more precise design of an oligonucleotide is required, and in the case of multiplex detection, the difficulty of the design of such an oligonucleotide is more highlighted. As a representative example, there is a technique for finding a conserved region from a number of target nucleic acid molecules having genetic diversity and designing oligonucleotides that hybridize to the conserved region (see Wang, D et al., Proc. Natl Acad. Sci. USA, 99:15687-15692(2002); Lin, F. M et al., IEEE Trans. Inf. Technol. Biomed., 10:705-713(2006); Chou, C. C et al., BMC Bioinform., 7:232(2006); Chizhikov, V et al., J. Clin. Microbiol., 40:2398-2407(2002)); Laassri, M et al., J. Virol. Methods, 112:67-78(2003) Mehlmann, M. et al, J. Clin. Microbiol., 44:2857-2862(2006)).

For the design of oligonucleotides, there are methods to consider together whether secondary structures such as helices or various types of loops are formed in a target nucleic acid, as well as primary structures (base sequences) for complementary binding between oligonucleotides and the target nucleic acid. Even though the oligonucleotide has excellent properties, when a thermodynamic stem-loop occurs at a specific site in the target nucleic acid, annealing or extension at the specific site is hindered, thereby reducing amplification efficiency, and as a result, there is a high possibility of failing to accurately detect the target nucleic acid.

Many methods are known for predicting the secondary structure of nucleic acids. As representative examples, there are an experimental method of predicting a secondary structure of a nucleic acid based on the thermodynamic principle that a folding phenomenon in which a nucleic acid sequence forms a secondary structure occurs in the lowest and stable energy state, an Knowledge-based method of analyzing a nucleic acid structure which has already been experimentally known and comparing the analyzed nucleic acid structure with a nucleic acid sequence of which a structure has not yet been known to predict a structure based on sequence similarity, and the like. Various software for predicting a secondary structure of a nucleic acid has been developed based on these various methods (see Rost B, Sander C, Schneider R, “PHD: An automatic mail server for protein secondary structure prediction”, Comput Appl Biosci, pp. 53-60, 1994).

However, conventional methods merely present analysis values, such as melting temperature and free energy, which are thermodynamic indices in a corresponding structure, as numerical values as a result of prediction of a secondary structure of a nucleic acid, and have a limitation in that they do not present a correlation on how much the secondary structure actually affects in the process of nucleic acid amplification. In addition, since the correlation between the secondary structure and the nucleic acid amplification reaction is not considered in the process of designing the oligonucleotide, there is a problem in that the accurate detection of the target nucleic acid using the oligonucleotide is likely to fail.

Throughout this specification, a number of cited and patent documents are referred to and cited. The cited literature and patent disclosures are incorporated herein by reference in their entirety to more clearly describe the level of the art to which the invention pertains and the contents of the invention.

Advantages and features of the present disclosure, and methods of achieving them will become apparent with reference to embodiments described below in detail together with the accompanying drawings. However, the present invention is not limited to the embodiments disclosed below, but may be implemented in various different forms, and only the embodiments are provided so that the disclosure of the present invention is complete and the scope of the present invention is fully known to those skilled in the art to which the present invention pertains, and the present invention is only defined by the scope of Claims.

In describing the embodiments of the present disclosure, when it is determined that a detailed description of a known function or configuration may unnecessarily obscure the subject matter of the present disclosure, the detailed description thereof will be omitted. Further, terms to be described below are terms defined in consideration of functions in the embodiments of the present disclosure, and may vary depending on the intention or custom of a user or an operator. Therefore, the definition should be made based on the contents throughout this specification.

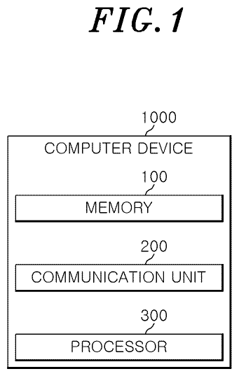

Prior to describing FIG. 1, terms used herein will be described.

As used herein, the term “nucleic acid sequence” refers to a target nucleic acid molecule as a specific nucleic acid sequence. In addition, a nucleic acid sequence means that bases, which are one of the components of a nucleotide, are arranged in order. For example, a nucleic acid sequence in the present disclosure may be used interchangeably with a base sequence. Each of the individual bases constituting the nucleic acid sequence may correspond to, for example, one of the four bases of A, G, C, and T.

The term “target analyte” may refer to various materials (e.g., biological materials and non-biological materials). The target analyte may specifically include at least one of a biological material, more specifically nucleic acid molecules (e.g., DNA and RNA), proteins, peptides, carbohydrates, lipids, amino acids, biological compounds, hormones, antibodies, antigens, metabolites, and cells.

The term “target analyte” or “organism” may include any form of organism or organism desired to be analyzed, obtained, or detected. For example, the organism may mean an organism belonging to one genus, species, subspecies, subtype, genotype, siro type, strain, isolate or cultivar. An organism and a target analyte in the present disclosure can be used interchangeably with each other.

The organism may include prokaryotic cells, eukaryotic cells, virus(es), or viroids. The virus may include, for example, a virus causing respiratory disease: influenza A virus (Flu A), influenza B virus (Flu B), respiratory syncytial virus A (RSV A), respiratory syncytial virus B (RSV B), Covid-19 virus, parainfluenza virus 1 (PIV 1), parainfluenza virus 2 (PIV 2), parainfluenza virus 3 (PIV 3), parainfluenza virus 4 (PIV 4), metapneumovirus (MPV), human enterovirus (HEV), human bocavirus (HBoV), human rhinovirus (HRV), coronavirus, adenovirus. The virus may include, for example, a virus causing gastrointestinal disease: norovirus, rotavirus, adenovirus, astrovirus, and sapovirus. As another example, the virus may include human papillomavirus (HPV), middle east respiratory syndrome-related coronavirus (MERS-CoV), dengue virus, herpes simplex virus (HSV), human herpes virus (HHV), epstein-barr virus (EMV), varicella zoster virus (VZV), cytomegalovirus (CMV), HIV, Parvovirus B19, Parechovirus, Mumps, Dengue virus, Chikungunya virus, Zika virus, West Nile virus, hepatitis virus, and poliovirus.

The organism according to an embodiment of the present disclosure may be GBS serotype, Bacterial colony, or v600e. The organism in the present disclosure may include not only the above-described virus, but also various analytes such as bacteria and human, and may be a specific site of a gene that is cleaved using CRISPR technology, and the scope of the organism is not limited by the above-described examples.

The term “sample” refers to a biological sample (e.g., cells, tissues, and body fluids) and a non-biological sample (e.g., food, water, and soil). The biological sample may include, for example, at least one of virus, bacteria, tissue, cells, blood (including whole blood, plasma, and serum), lymph, bone marrow, saliva, sputum, swab, aspiration, milk, urine, stool, eye fluid, semen, brain extract, spinal fluid, joint fluid, thymus fluid, bronchial lavage fluid, ascites, and amniotic fluid. This sample may or may not include the target analyte described above.

Meanwhile, the target analyte, particularly the target nucleic acid molecule, may be amplified by various methods as described above. The amplification reaction in the present disclosure may be by PCR, LCR, SDA, TMA, NASBA, RCA, Q-Beta Replicase, LAMP, or RPA.

According to an embodiment, the amplification reaction for amplifying the signal indicating the presence of the target analyte may be performed in a manner in which the target analyte is amplified, and the signal is also amplified (e.g., a real-time PCR method). Alternatively, according to an embodiment, the amplification reaction may be performed in a manner in which the target analyte is not amplified, but only a signal indicating the presence of the target analyte is amplified (e.g., CPT method). As such, the amplification reaction may be accompanied by a signal change, and thus, the degree of progress of the amplification reaction may be evaluated by measuring the signal change. As such a signal providing means, a signal generation composition including a label itself or an oligonucleotide linked to the label may be used. Various methods (e.g., a TaqMan™ probe method, a molecular beacon method, etc.) of generating a signal indicating the presence of a target analyte using the signal generation composition are known.

Here, the term “signal” means a measurable output. In addition, the measured magnitude or change of the signal serves as an indicator to qualitatively or quantitatively indicate the properties of the target analyte, specifically the presence or absence of the target analyte in the sample. Examples of the indicator include, but are not limited to, fluorescence intensity, luminescence intensity, chemiluminescence intensity, luminescence intensity, phosphorescence intensity, charge transfer, voltage, current, power, energy, temperature, viscosity, light scatter, radioactivity intensity, reflectivity, light transmittance, and absorbance.

The term “dataset” refers to a result of an amplification reaction for a target analyte in a sample. Specifically, the dataset refers to data obtained from an amplification reaction for a target analyte in a sample or data processed from the corresponding data. The dataset obtained through the amplification reaction may include an amplification cycle.

Here, the term “cycle” refers to a unit of change in a condition in a plurality of measurements accompanied by a change in the condition. The change of the predetermined condition means, for example, an increase or decrease in temperature, reaction time, the number of reactions, concentration, pH, the number of replicates of a measurement object (e.g., nucleic acid), etc. Thus, a cycle may be a time or process cycle, a unit operation cycle, and a reproductive cycle.

More specifically, the term “cycle” means one unit of the above repetition when a reaction of a certain process is repeated, or a reaction is repeated based on a certain time interval. For example, in the case of a nucleic acid amplification reaction, one cycle means a reaction including denaturation of a nucleic acid, annealing of a primer, and extension of a primer. In this case, the change in the predetermined condition is an increase in the number of repetitions of the reaction, and a repeating unit of the reaction including the series of steps is set as one cycle.

Meanwhile, the dataset obtained from the amplification reaction includes a plurality of data points including cycles of the amplification reaction and signal values at the cycles.

Here, the term “signal value” means a value obtained by digitizing a level (e.g., intensity of a signal) of a signal actually measured in a cycle of an amplification reaction according to a certain scale or a modified value thereof. The deformation value may include a mathematically processed signal value of the actually measured signal value (i.e., the signal value of the raw dataset), and may include, for example, a logarithmic value or derivatives.

The term “data point” means one coordinate value including a cycle and a signal value. In addition, the term “data” means all information constituting the dataset. For example, each of the cycle of the amplification reaction and the signal value may correspond to data. The data points obtained by the amplification reaction may be represented by coordinate values that can be represented in a two-dimensional orthogonal coordinate system. In the coordinate value, the X-axis represents the number of corresponding cycles, and the Y-axis represents a signal value measured or processed in a corresponding cycle.

The term “dataset” refers to a set of the data points. For example, the dataset may be a collection of data points directly obtained through an amplification reaction performed in the presence of a signal generating composition, or may be a dataset obtained by modifying such a dataset. The dataset may be a part or all of a plurality of data points obtained by an amplification reaction or modified data points thereof.

The dataset may include a dataset obtained by processing a plurality of datasets. When analysis of a plurality of target analytes is performed in one reaction vessel, the data sets for the plurality of target analytes may be obtained through processing of the data sets obtained from the reaction performed in the one reaction vessel. For example, a dataset for a plurality of target analytes made in one reaction vessel may be obtained by processing a plurality of datasets obtained from signals measured at different temperatures. For example, methods for detecting signals generated at different detection temperatures using a single type of detector in order to detect two target nucleic acid sequences in a sample (see Korean Patent No. 10-2050601) are known in the art.

According to an embodiment, the dataset may be a raw dataset obtained from the detection device, a mathematically modified dataset of the raw dataset, a normalized dataset of the raw dataset, or a standardized dataset of the mathematically transformed dataset.

Here, the raw dataset refers to a dataset including a signal value directly obtained from an amplification reaction. For example, the raw data set includes a set of signal values obtained from a detection device in which an amplification reaction for detecting a target analyte has been performed, subjected to basic signal processing on the detection device, and then passed to the signal analysis step.

In addition, a mathematically modified dataset of the raw dataset refers to a dataset converted from the raw dataset through mathematical processing. For example, the mathematically processed dataset may be a dataset obtained by removing at least a portion of the background signal from the raw dataset, that is, a baseline-subtracted dataset. The baseline subtracted dataset may be obtained by various methods known in the art (e.g., U.S. Pat. No. 8,560,247). As another example, the mathematically processed dataset may be a dataset obtained by removing at least some background signals and noise signals due to noise, interference, or the like from the raw dataset.

Further, normalization refers to a process of reducing or eliminating a signal deviation between data sets for a plurality of reactions. Standardization is an aspect of correction or adjustment that corrects, modifies, etc., data (in particular, signal values) of a dataset for analysis purposes.

In an embodiment, the dataset includes 200 or fewer, 150 or fewer, 100 or fewer, 50 or fewer, 40 or fewer, and 30 or fewer data points. In an embodiment, the dataset includes two or more, five or more, ten or more, and twenty or more data points. The dataset may be plotted, whereby an amplification curve may be obtained.

The term “oligonucleotide” as used herein refers to natural or modified monomers or a linear oligomer of linkages, which includes deoxyribonucleotides and ribonucleotides, which may specifically hybridize to a target nucleic acid sequence, and which may be naturally present or artificially synthesized. For example, the binding oligonucleotide may be used interchangeably with a primer or a probe.

The term “primer” refers to an oligonucleotide that is able to serve as an initiation point for synthesis under conditions in which synthesis of a primer extension product is induced (i.e., under conditions in which a nucleotide and a polymerization agent such as a DNA polymerase present and temperature and pH is suitable). In addition, the term “probe” refers to a single-stranded nucleic acid molecule including a region or regions complementary to a target nucleic acid sequence. In addition, the probe may include a marker capable of generating a signal for target detection.

The oligonucleotide may have a conventional primer and probe structure composed of a sequence hybridized with the target nucleic acid sequence. Alternatively, it may be an oligonucleotide having a unique structure by modifying the structure of the oligonucleotide. For example, the oligonucleotide may have the structure of a Scorpion primer, a Molecular Beacon probe, a Sunrise primer, a High Beacon probe, a tagging probe, a DPO primer or probe (WO 2006/095981), and a PTO probe (see WO 2012/096523).

The set of oligonucleotides may mean one or more oligonucleotides, and may be interpreted to mean a sequence set of oligonucleotides including a forward sequence and a reverse sequence according to an embodiment. In an embodiment, the oligonucleotide set may include a primer set of a forward primer and a reverse primer. As an example, the forward primer is a primer that anneals to the antisense strand, the non-coding strand, or the template strand, and may be a primer that serves as a starting point for the coding or positive strand of the target analyte. In addition, the reverse primer is a primer that anneals to the 3′ end of the sense strand or coding strand, and may be a primer that serves as a starting point for synthesizing the complementary strand of the coding sequence or the non-coding sequence of the target analyte. Here, the forward primer and the reverse primer described above may refer to a pair primer that determines a specific amplification region in the target nucleic acid sequence, and in some embodiments, may refer to each primer that does not operate as a pair.

FIG. 1 schematically illustrates a block diagram of a computer device 1000 according to an embodiment.

Referring to FIG. 1, the computer device 1000 may include a memory 100, a communication unit 200, and a processor 300. The configuration of the computer device 1000 shown in FIG. 1 is only an example shown briefly. In an embodiment, the computer device 1000 may include other components for performing a computing environment of the computer device 1000, and only some of the disclosed components may constitute the computer device 1000.

The computer device 1000 may mean a node configuring a system for implementing embodiments of the present disclosure. The computer device 1000 may include any type of user terminal and/or any type of server.

A user terminal may include any form of terminal that is interactable with a server or other computing device. The user terminal may include, for example, a mobile phone, a smartphone, a notebook computer, personal digital assistants (PDAs), a slate PC, a tablet PC, and an ultrabook.

The server may include any type of computing system or computing device such as, for example, a microprocessor, a mainframe computer, a digital processor, a portable device, and a device controller. In an embodiment, the server may include an entity that stores and manages a plurality of datasets. The server may include a storage unit (not shown) for storing data for learning of an estimation model to be described later, and the storage unit may be included in the server or may exist under the management of the server. As another example, the storage may be implemented in a form that exists outside the server and is capable of communicating with the server. In this case, the storage unit may be managed and controlled by another external server different from the server.

The computer device 1000 may perform technical features according to embodiments to be described later. For example, the computer device 1000 may provide a prediction result of an amplification reaction affected by an nth-order structure in the amplification reaction for a nucleic acid sequence by at least partially using the analysis data for the nth-order structure of the nucleic acid sequence.

The memory 100 may store at least one instruction executable by the processor 300. In an embodiment, the memory 100 may store any form of information generated or determined by the processor 300 and any form of information received by the computer device 1000. In an embodiment, the memory 100 may be a storage medium storing computer software that allows the processor 300 to perform operations according to embodiments of the present disclosure. Accordingly, the memory 100 may mean computer-readable media for storing software codes, data to be executed as a code, and an execution result of the code, which are necessary to perform the embodiments of the present disclosure.

In an embodiment, the memory 100 may refer to any type of storage medium, and for example, the memory 100 may include at least one type of storage medium among a flash memory type, a hard disk type, a multimedia card micro type, a card type memory (e.g., SD or XD memory), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computer device 1000 may operate in association with a web storage performing a storage function of the memory 100 on the Internet. The above description of the memory is merely an example, and the memory 100 of the present disclosure is not limited thereto.

The communication unit 200 may be configured regardless of a communication aspect such as wired and wireless communication, and may be configured by various communication networks such as a personal area network, a wide area network, and the like. In addition, the communication unit 200 may operate based on the known World Wide Web, and may use a wireless transmission technology used for short range communication such as Infrared Data Association (IrDA) or Bluetooth. For example, the communication unit 200 may be responsible for transmitting and receiving data required to perform a technique according to an embodiment of the present disclosure.

The processor 300 may perform technical features according to embodiments to be described later by executing at least one instruction stored in the memory 100. In an embodiment, the processor 300 may be configured with at least one core, and may include a processor for data analysis and/or processing such as a central processing unit (CPU), a general purpose graphics processing unit (GPGPU), a tensor processing unit (TPU), or the like of the computer device 1000.

The processor 300 according to an embodiment of the disclosure may perform an operation for learning. In an embodiment, the processor 300 may perform calculations for learning such as processing of an independent variable used for a prediction function, calculation of a dependent variable using the independent variable, error calculation, weight update, and the like in machine learning. In another embodiment of the disclosure, the processor 300 may perform computation for learning of neural networks in deep learning, such as processing input data for learning, extracting features in the input data, error computation, weight update of the neural network using backpropagation, and the like. At least one of the CPU, the GPGPU, and the TPU of the processor 300 may process an operation for training. For example, the CPU and the GPGPU may train a prediction function or a network function together, and may process data classification using the trained function. In addition, in the exemplary embodiment of the present disclosure, the processors of the plurality of computing devices may be used together to process the training of the prediction function or the network function and the data classification using the trained function. In addition, the computer program executed in the computing device according to the exemplary embodiment of the present disclosure may be a CPU, GPGPU, or TPU executable program.

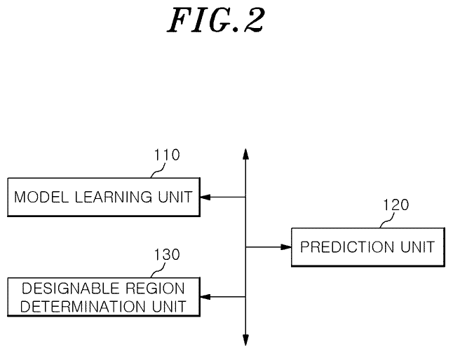

Various technical features performed by the processor 300 will be described with reference to FIG. 2.

FIG. 2 is a diagram illustrating modularized software implemented by the computer device 1000 shown in FIG. 1. Referring to FIG. 2, software implemented as the processor 300, which is hardware, executes at least one instruction stored in the memory 100 may be modularized into at least one of a model learning unit 110, a prediction unit 120, and a designable region determination unit 130. For example, each of the model learning unit 110, the prediction unit 120, and the designable region determination unit 130 may be implemented as a computer program, and instructions and data for execution thereof may be stored in the memory 100 and executed by the processor 300, but are not limited thereto. According to an embodiment, the instructions and data for execution of the model learning unit 110 may be stored in a memory of a server among a plurality of entities implementing the computer device 1000 and executed in the server. In addition, instructions and data for execution of each of the prediction unit 120 and the designable region determination unit 130 may be stored in the memory of the user terminal among the plurality of entities and executed in the user terminal.

The model learning unit 110 according to an embodiment of the present disclosure may be implemented to obtain a prediction model that provides a prediction result of a nucleic acid amplification reaction affected by an nth-order structure. In an embodiment, the prediction model may include an artificial intelligence-based model.

In the present specification, the prediction model according to an embodiment may mean any form of computer program operating based on at least one of one or more functions, a network function, an artificial neural network, and a neural network. According to example embodiments, a model, a function, a network function, a neural network, and a neural network may be used interchangeably. The function may represent a correlation between one or more independent variables and one or more dependent variables, and define how they operate. In the neural network, one or more nodes are interconnected through one or more links to form an input node and an output node relationship within the neural network. The characteristics of the neural network may be determined according to the number of nodes and links in the neural network, the correlation between the nodes and the links, and the weight assigned to each of the links. In an embodiment, the neural network may be various types of neural networks that are known.

The prediction model according to an embodiment may include at least one of a regression model and a classification model.

Here, the regression analysis model refers to an analysis model for obtaining a model between a plurality of variables with respect to continuous variables and then measuring the goodness-of-fit. The regression analysis model according to an embodiment may be implemented based on (a) a simple regression analysis method for analyzing a relationship between one dependent variable and one independent variable and/or (b) a multiple regression analysis method for analyzing a relationship between one dependent variable and multiple independent variables. Hereinafter, embodiments to which a prediction model based on the multiple regression analysis method is applied are mainly illustrated, but are not limited thereto.

In an embodiment, the regression analysis model may include at least one of a linear regression analysis model, a non-linear regression analysis model, a decision tree model, and an ensemble model. In an embodiment, the linear regression analysis model may include at least one of a Robust, a Lasso, and a Ridge. In addition, the linear regression analysis model may include at least one of a normal equation, a least squares method, and a gradient descent method as a classification according to a method of obtaining the optimal solution. The gradient descent method may include a stochastic gradient descent (SGD), a batch gradient descent (BGD), and the like. The non-linear regression analysis model may include at least one of a convolutional neural network (CNN), a recurrent neural network (RNN), and a deep neural network (DNN). In addition, the ensemble model is a method of deriving a final result from results of various models, and may include at least one of a bagging method for reconstructing data for diversification of the model, a random forest method for reconstructing data and variables for diversification of the model, a boosting method for training by assigning a weight to data having a large error in previous training, and a stacking method for using an output of the model as a new independent variable. As an example, the boosting series may include a gradient boosting algorithm (GBM), AdaBoost, XGBoost, LightGBM, and the like.

In addition, the classification model refers to an analysis model that independently determines a label (e.g. category) of new data by analyzing the relationship with the label specified in the training data. The classification model according to an embodiment may be classified into a binary classification model or a multi-label classification model according to the number of categories to be classified. The classification model according to an embodiment may include at least one of a logistic regression model, a linear discriminant analysis (LDA) model, a K-nearest neighbor (KNN) model, a Naive Bayes model, a decision tree model, an ensemble model, and a support vector machine (SVM) model. For example, the logistic regression model may include Softmax regression. According to an embodiment, the classification model may further include algorithms described as examples of the above-described regression analysis model.

However, the description of the prediction model described above is merely an example, and the prediction model in the present disclosure is not limited thereto. For example, the prediction model may be a machine learning model or a deep learning model in various forms known in the art. A detailed embodiment of the prediction model will be described later.

The prediction model according to an embodiment may be learned by at least one of supervised learning, unsupervised learning, semi-supervised learning, self-supervised learning, and reinforcement learning. The learning may be a process in which a machine learning model or a deep learning model applies knowledge for performing a specific operation to a prediction function or a neural network.

The prediction model according to an embodiment may be learned in a direction of minimizing an error of an output. For example, a series of processes for (a) inputting the training data to the prediction model, (b) calculating an error between an output of the prediction model for the training data and the expected output, and (c) updating an operation method of the prediction model in order to reduce the error, and/or (d) backpropagating the error from the output layer toward the input layer to update the weight of each node may be repeatedly performed. In supervised learning, labeled data in which the correct answer is labeled on each training data may be used, and in unsupervised learning, unlabeled data in which the correct answer is not labeled on each training data may be used. According to an embodiment, the amount of change in the updated weight may be determined according to a training rate. The calculation of the prediction model for the input data and the update of the error may constitute a learning cycle (epoch), and the learning rate may be applied differently according to the repetition number of the epoch.

The model learning unit 110 according to an embodiment may train a prediction model to predict an amplification reaction result affected by an nth-order structure in the amplification reaction for a given nucleic acid sequence. Specifically, when a predetermined nucleic acid sequence and/or analysis data for an nth-order structure existing in the predetermined nucleic acid sequence is given, the model learning unit 110 may train the prediction model to predict a level at which a result of an amplification reaction for the nucleic acid sequence is affected by a formation of the nth-order structure in the nucleic acid sequence (or whether affected or not). Throughout the specification, the “affect” (or effect) may be broadly interpreted as a meaning including a change in efficiency of the amplification reaction or an inhibition of the amplification reaction, which occurs as amplification is delayed or inhibited in the process of the amplification reaction.

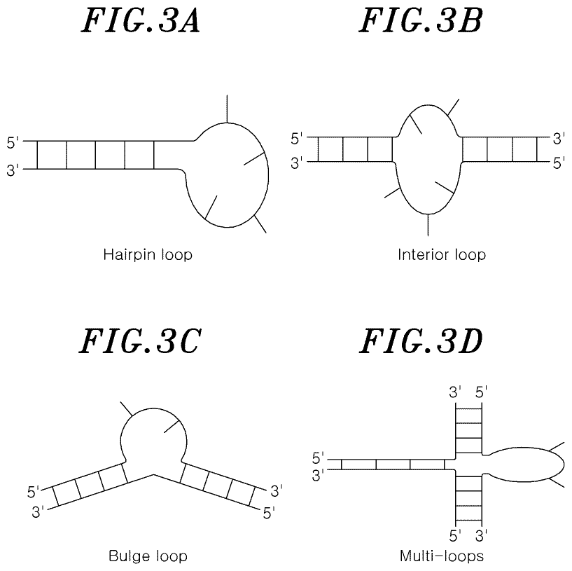

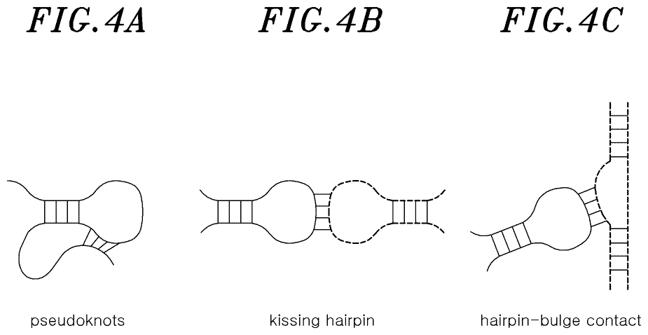

Meanwhile, n in the above-described nth-order structure is an integer not less than 2. In an embodiment, the nth-order structure may be at least one of a secondary structure, a tertiary structure and a quaternary structure. In an embodiment, n may be 2. In another embodiment, n may be 3. In another embodiment, n may be 4. This will be described with reference to FIGS. 3 and 4.

FIGS. 3A-3D show a diagram illustrating types of secondary structures of nucleic acid sequences according to an embodiment. Referring to FIGS. 3A-3D, the nth-order structure according to an embodiment may be a secondary structure representing interaction between two or more bases included in a nucleic acid sequence. In an embodiment, the secondary structure may include at least one of a hairpin loop, an internal loop, a bulge loop, multi-loops, a G-quadruplex, and combinations thereof. As an example of the secondary structure, the hairpin loop is illustrated in FIG. 3A, the internal loop is illustrated in FIG. 3B, the bulge loop is illustrated in FIG. 3C, and the multi-loops is illustrated in FIG. 3D. In an embodiment, the G-quadruplex may be formed from a nucleic acid sequence containing a relatively large amount of G among the bases. For example, the G-quadruplex may include G-tetrads that are formed in a spiral shape and are formed from 1, 2, or 4 strands.

FIGS. 4A-4C a diagram illustrating types of tertiary structures of nucleic acid sequences according to an embodiment. Referring to FIGS. 4A-4C, the nth-order structure according to an embodiment may be a tertiary structure having two or more interactions described above, and may include, for example, a case of folding by hydrogen bonding in a state in which there is secondary structure, and the like. In an embodiment, the tertiary structure may include at least one of a pseudoknot, a kissing hairpin, and a hairpin-bulge contact. As an example of the third structure, the pseudoknot is illustrated in FIG. 4A, the kissing hairpin is illustrated in FIG. 4B, and the hairpin-bulge contact is illustrated in FIG. 4C.

Hereinafter, although the hairpin loop of the secondary structure is mainly described as an embodiment of the nth-order structure in the specification, the nth-order structure of the present disclosure is not limited thereto. According to an embodiment, the nth-order structure may include various types of structures known in the art in addition to the above-described embodiments, may include two or more of the secondary structure to the quaternary structure, and may include a fifth-order structure or more, and is not limited to anyone.

The model learning unit 110 according to an embodiment may obtain a plurality of training data for learning of a prediction model. In an embodiment, each set of training data may include a training input data and a training answer data corresponding to the training input data. In an embodiment, the training input data may mean data (e.g., a value of an independent variable) input to the prediction model in supervised learning, and the training answer data may mean data labeled as a correct answer in supervised learning (e.g., an expected output of a dependent variable or a label determined from the expected output). Meanwhile, the terms of ‘training data’, ‘a set of training data’ and ‘training dataset’ in the present disclosure can be used interchangeably with each other. For example, the plurality of training data means a plurality of sets of training data (or a plurality of training datasets).

The training input data according to an embodiment may include a first analysis data for an nth-order structure that present or may be present in a predetermined nucleic acid sequence. According to an embodiment, the training input data may further include at least one of a nucleic acid sequence, an amplicon sequence and an oligonucleotide sequence, a melting temperature (Tm), a length, a type, and a GC content. The training answer data according to an embodiment may include an amplification reaction result for the corresponding nucleic acid sequence. The above-described amplification reaction result for the nucleic acid sequence represents (i) a level (or an extent) at which the amplification reaction for the corresponding nucleic acid sequence is affected by a formation of the nth-order structure in the nucleic acid sequence and/or (ii) whether the amplification reaction for the corresponding nucleic acid sequence is affected or not by the formation of the nth-order structure in the nucleic acid sequence. Specific embodiments of such training data will be described again later.

The model learning unit 110 according to an embodiment may train a prediction model using the plurality of training data. For example, the model learning unit 110 may input the training input data in each training data to the prediction model, update the prediction model by using an output obtained from the prediction model and the training answer data in each training data, and train the prediction model by repeating this process for each of the plurality of training data.

In an embodiment, when the prediction model is implemented as the machine learning model based on the multiple regression analysis method, each feature (e.g., the first analysis data for the nth-order structure) included in the training input data may correspond to each independent variable of the prediction function based on the multiple regression analysis method, and the training answer data (e.g., an amplification inhibition level) may correspond to the expected output of the dependent variable of the corresponding prediction function. In the supervised learning process for the prediction model, the model learning unit 110 may feedback the difference between the output of the dependent variable by the independent variables of the predictive function and the expected output to update the operating method of the prediction model in a direction to reduce the difference.

In another embodiment, when the prediction model is implemented as a DNN-based deep learning model, the training input data may be pre-processed in a form of a predetermined vector or matrix and provided to the neural network input layer of the prediction model. The prediction model may output a plurality of probability values for each class based on features extracted from the training input data through successive layers of the deep neural network. In the supervised learning process for the prediction model, the model learning unit 110 may update weights of the prediction model so that the difference between the output and the training answer data is minimized.

According to an embodiment of the present disclosure, the prediction unit 120 may be implemented to obtain a prediction result of an amplification reaction affected by an nth-order structure in an amplification reaction for a target nucleic acid sequence by using the trained prediction model. Specifically, the prediction unit 120 may obtain input data comprising a second analysis data for an nth-order structure of the target nucleic acid sequence, may provide the input data to the prediction model, and may obtain a prediction result of an amplification reaction affected by the nth-order structure in the amplification reaction for the target nucleic acid sequence from the prediction model. Throughout the specification, the term “nucleic acid sequence” may be understood as a term referring to a nucleic acid sequence that becomes a target of learning of the prediction model in a learning process of the model (e.g., by the server), and the term “target nucleic acid sequence” may be understood as a term referring to a nucleic acid sequence that becomes a target of prediction in a process of obtaining a prediction result by using the trained prediction model (e.g., by the user terminal).

According to an embodiment of the present disclosure, the designable region determination unit 130 may be implemented to determine a designable region of an oligonucleotide based at least in part on the obtained prediction result. Here, the designable region may mean a region used when designing an oligonucleotide for detecting a specific target analyte among regions each having a plurality of successive bases. In an embodiment, when the amplification reaction is affected by the nth-order structure according to the prediction result, the designable region determination unit 130 may exclude a region corresponding to the target nucleic acid sequence from the candidate regions that can be selected as the designable region.

Various embodiments of the operations/functions of the model learning unit 110, the prediction unit 120, and the designable region determination unit 130 presented above will be described in more detail below.

### Various Embodiments of Training Data

First, after various embodiments of the training data are described, embodiments of a process of obtaining the training data, a learning process of a prediction model using the training data, and a prediction process using the trained prediction model will be described.

The training data to an embodiment may include a first analysis data for an nth-order structure present in a predetermined nucleic acid sequence. Here, the first analytical data of the nth-order structure means analytical data directly or indirectly representing whether the nth-order structure is formed in the corresponding nucleic acid sequence or the possibility of formation thereof. For example, the first analysis data for the nth-order structure may include thermodynamic stability when a specific secondary structure (e.g., hairpin loop) is formed by interaction between bases in the corresponding nucleic acid sequence under an environment in which a predetermined heat is applied, entropy or energy of a thermodynamic system when the specific secondary structure is formed, and the like.

In an embodiment, the first analytical data for the nth-order structure may include a thermodynamic data for a formation of the nth-order structure in the corresponding nucleic acid sequence. Here, the thermodynamic data means data for one or more thermodynamics properties. The thermodynamic properties may be thermodynamic parameters used to predict the thermodynamic structure of a nucleic acid sequence based on the thermodynamic principle that a folding phenomenon for forming an nth-order structure in the nucleic acid sequence occurs in a low and stable energy state. Here, the thermodynamic properties may be understood as meaning including various properties such as Gibbs free energy, Gibbs free entropy, internal energy, enthalpy, entropy, and the like.

In an embodiment, the thermodynamic data may be indicated as a change in thermodynamic free energy. In an embodiment, the thermodynamic free energy may include a Gibbs free energy, but is not limited thereto, and may include various other energies such as, for example, internal energy, Helmholtz free energy, and specific internal entropy. As an example, the thermodynamic data may include the amount of change (ΔG) in Gibbs free energy when hairpin loop is formed in a corresponding nucleic acid sequence in a system under certain conditions (e.g., temperature, pressure). Hereinafter, the thermodynamic data is mainly illustrated as the amount of change in Gibbs free energy, but is not limited thereto, and may include the size or intensity of various thermodynamic properties described above or known in the art.

In an embodiment, the thermodynamic data for the formation of the nth-order structure may be thermodynamic data for a formation of an arbitrary nth-order structure, thermodynamic data for a formation of a specific nth-order structure, or thermodynamic data for a formation of each of a plurality of nth-order structures. For example, the thermodynamic data for the formation of the nth-order structure may include ΔG when any one or more of the types of the preset nth-order structures in the nucleic acid sequence are formed, may include ΔG when the hairpin loop is formed, or may include all of ΔG when the hairpin loop is formed, ΔG when the internal loop is formed, ΔG when the bulge loop is formed, ΔG when the multi-loops is formed, and ΔG when the G-quadruplex is formed.

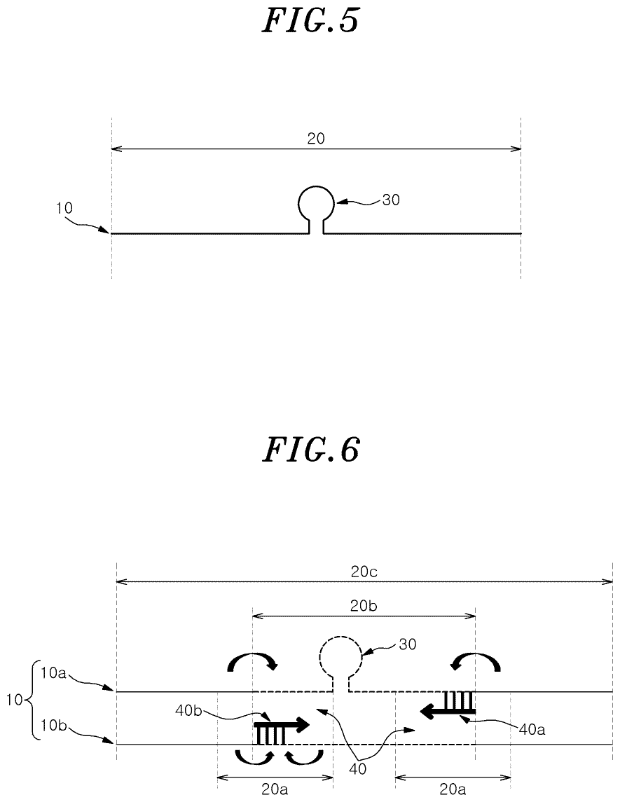

FIG. 5 is a diagram for exemplarily describing thermodynamic data for an nth-order structure present in a nucleic acid sequence according to an embodiment.

Referring to FIG. 5, the first analytic data for the nth-order structure may include thermodynamic data for the nth-order structure present within the range of nucleic acid sequences. Such thermodynamic data may include, for example, the amount of change in Gibbs free energy (ΔG) for a secondary structure (see identification number 30) (e.g., hairpin) formed in a nucleic acid sequence within the entire range (see identification number 20) of the nucleic acid sequence (see identification number 10) including a series of bases. As an example, the smaller the amount of change in Gibbs free energy (ΔG) (e.g., the greater the negative value), the greater the thermodynamic stability of the corresponding secondary structure, and it may mean that there is a high possibility that the corresponding secondary structure is formed in the amplification reaction.

FIG. 6 is a diagram for exemplarily describing the first to the third thermodynamic data for an nth-order structure in which a process of an amplification reaction for a nucleic acid sequence is considered, according to an embodiment.

Referring to FIG. 6, the first analytical data for the nth-order structure may include at least one of a plurality of thermodynamic data for the nth-order structure present in different ranges in the nucleic acid sequence. In an embodiment, each of the plurality of thermodynamic data may refer to thermodynamic data for when an nth-order structure present in a predetermined range in the nucleic acid sequence in one or more specific steps among step denaturation, step annealing, and step extension included in each cycle in the process of an amplification reaction for the nucleic acid sequence.

The plurality of thermodynamic data according to an embodiment may include at least one selected from the group consisting of thermodynamic data of a first block unit 20a, thermodynamic data of a second block unit 20b, and thermodynamic data of a third block unit 20c.

Here, the thermodynamic data of the first block unit 20a indicates thermodynamic data of an nth-order structure present in the first block unit 20a. The first block unit 20a may be defined as a predetermined range based on a region in which an oligonucleotide (e.g., a primer) is bound to a corresponding nucleic acid sequence during the annealing step of the amplification reaction. For example, the thermodynamic data of the first block unit 20a may include the amount of change in a first Gibbs free energy (ΔG₁) of the secondary structure (see identification number 30) formed in a region (see identification number 20a), and the region (see identification number 20a) may be within a predetermined distance from a region (see identification number 20a) where a primer sequence (see identification number 40) is annealed to the nucleic acid sequence among the entire range (see identification number 20c) of the nucleic acid sequence (see identification number 10). The primer sequence (see identification number 40) may be a sequence designed to bind to a specific region in the corresponding nucleic acid sequence. In an embodiment, the thermodynamic data of the first block unit 20a may be divided into thermodynamic data for each of a forward oligonucleotide sequence and a reverse nucleotide sequence. For example, the amount of change in the first Gibbs free energy (ΔG₁) may include an amount of change in a Gibbs free energy (ΔG_{1A}) of the forward primer sequence (see identification number 40a) and an amount of change in a Gibbs free energy (ΔG_{1B}) of the reverse primer sequence (see identification number 40b).

In an embodiment, the first block unit 20a may include a region corresponding to a primer sequence, and the thermodynamic data of the first block unit 20a may be calculated in consideration of a predetermined temperature condition and a corresponding partial sequence in order to consider a case in which a secondary structure is formed around a binding region of a nucleic acid sequence and a primer. As an example, as the amount of change in the first Gibbs free energy (ΔG₁) is smaller, it may mean that a corresponding secondary structure is highly likely to be formed near a region bound to the primer sequence during the annealing step of the amplification reaction, and it may mean that the reaction is highly likely to be inhibited in the annealing step.

Also, the thermodynamic data of the second block unit 20b indicates thermodynamic data of an nth-order structure present in the second block unit 20b. The second block unit 20b may be defined as a range of a region to be extended by an oligonucleotide (e.g., a primer) bound to the corresponding nucleic acid sequence during an extension step of the amplification reaction. For example, the thermodynamic data of the second block unit 20b may include the amount of change in a second Gibbs free energy (ΔG₂) of the secondary structure (see identification number 30) formed in a region (see identification number 20b) where the primer sequence (see identification number 40) is annealed among the entire range (see identification number 20c) of the nucleic acid sequence (see identification number 10) and then extended.

In one embodiment, the second block unit 20b may include a region corresponding to a amplicon sequence representing a product generated by amplification in each cycle during an amplification reaction, and the thermodynamic data of the second block unit 20b may be calculated in consideration of the corresponding partial sequence and a predetermined temperature condition in order to consider the case where a secondary structure is formed in the amplicon region which is a product of the nucleic acid amplification reaction. As an example, as the amount of change in the second Gibbs free energy (ΔG₂) is smaller, it may mean that a corresponding secondary structure is more likely to be formed in the extension region during the extension step of the amplification reaction, and it may mean that the reaction may be more likely to be inhibited in the extension step.

Also, the thermodynamic data of the third block unit 20c indicates thermodynamic data of an nth-order structure present in the third block unit 20c. The third block unit 20c may be defined as a sequence comprising (i) the second block and (ii) an additional sequence at the 5′ end and the 3′ end of the second block. For example, when the length of the corresponding nucleic acid sequence is 1000 bp and the second block unit 20b is 200 bp, the third block unit 20c may be (i) a region of 200 bp corresponding to the second block unit 20b, (ii-1) a region of 1000 bp corresponding to the corresponding nucleic acid sequence, and (ii-2) a region of about 500-800 bp obtained by adding a region of additional sequences between the second block unit 20b and the corresponding nucleic acid sequence (e.g., a non-overlapping region between the identification numbers 20b and 20c) to the region of the second block unit 20b. In an embodiment, the thermodynamic data of the third block unit 20c may be thermodynamic data of an nth-order structure present within a range of a corresponding nucleic acid sequence affecting during the annealing step or the extension step before amplicon formation during the amplification reaction. For example, the thermodynamic data of the third block unit 20c may include the amount of change in a third Gibbs free energy (ΔG₃) of the secondary structure (see identification number 30) formed in the entire range (see identification number 20c) of the nucleic acid sequence (see identification number 10).

In an example embodiment, the third block unit 20c may include a region corresponding to the nucleic acid sequence, and the thermodynamic data of the third block unit 20c may be calculated in consideration of the partial sequence and a predetermined temperature condition in order to consider a case in which a secondary structure is formed in the entire region of the nucleic acid sequence before a product of an amplification reaction is generated, for example, in an initial stage of PCR amplification. As an example, as the amount of change in the third Gibbs free energy (ΔG₃) is smaller, it means that there is a high possibility that a corresponding secondary structure is formed in a nucleic acid sequence in the initial stage of amplification during the amplification reaction, and it may mean that there is a high possibility that the reaction may be inhibited in the initial stage of amplification.

Meanwhile, FIG. 6 illustrates a case in which an oligonucleotide sequence (see identification number 40) is bound to a nucleic acid sequence (see identification number 10) separated into a single strand during the annealing step of the amplification reaction. In an embodiment in which the type of the nucleic acid sequence is DNA, identification numbers 10a and 10b may be understood to exemplify a template DNA sequence functioning as a template and a complementary DNA sequence for the template, respectively. In an embodiment where the type of nucleic acid sequence is RNA, identification numbers 10a and 10b may be understood to exemplify a RNA sequence being the target and a complementary sequence for the RNA sequence, respectively. In addition, oligonucleotide sequences denoted by identification number 40 are designed to bind to each single-stranded nucleic acid sequence, and it may be understood that they exemplify a primer set including, for example, a forward primer (see identification number 40a) and a reverse primer (see identification number 40b).

Meanwhile, in another embodiment, the first analysis data for the nth-order structure may include at least one of a value of n, a type of the nth-order structure, a characteristic of the nth-order structure, and information about whether the nth-order structure is formed. For example, the characteristics of the nth-order structure may include a pattern of a sequence in which a specific nth-order structure is mainly formed, a pattern of a GC content indicating a ratio of inclusion of G (guanine) or C (cytosine) among bases of a corresponding sequence, or the like. In addition, the information about whether the nth-order structure is formed may include, for example, a probability of forming the nth-order structure in the corresponding nucleic acid sequence calculated through a pre-stored equation, a frequency of forming the nth-order structure measured through experiments, and/or whether the nth-order structure is formed.

The first analysis data for the nth-order structure described above may include analysis data for the structural characteristics of a specific nth-order structure appearing at bases in the nucleic acid sequence. In an embodiment, the first analysis data may include analysis data for the structural characteristics of the G-quadruplex appearing at bases included in the nucleic acid sequence. For example, the first analysis data may include a G-quadruplex score (e.g., G4 hunter value) that quantifies G-richness and/or G-skewness based on the position, number, and/or distribution of G in the corresponding base sequence. By way of example, the G-quadruplex score may be interpreted as having structural characteristics that make it relatively easy to form the G-quadruplex as the value increases (or is above a predetermined threshold). In another embodiment, the first analysis data may include analysis data for the structural characteristics of any one of the hairpin loop, the internal loop, the bulge loop, the multi-loop, the pseudoknot, the kissing hairpin, and the hairpin-bulge contact appearing at bases included in the nucleic acid sequence. For example, the first analysis data may include a characteristic score that quantifies the degree to which the number or aspect of a specific base combination (e.g., the number of patterns in which consecutive G is more than a predetermined number) that is mainly seen for each specific type of the nth-order structure appears within the corresponding base sequence.

The training data according to an embodiment may further include at least one of the nucleic acid sequence, an amplicon sequence, and an oligonucleotide sequence. Here, the amplicon sequence is a sequence obtained from the corresponding nucleic acid sequence, and refers to a base sequence of amplicon indicating a product generated by amplification in each cycle during the amplification reaction for the nucleic acid sequence. In addition, the oligonucleotide sequence refers to a base sequence of an oligonucleotide (e.g., a primer) that is bound to the corresponding nucleic acid sequence and represents a material that mediates the amplification reaction. Depending on the embodiment, the type and order of bases included in the targeted nucleic acid sequence may be used itself as the training input data.

The training data according to an embodiment may further include information about a melting temperature (Tm) of at least one of the nucleic acid sequence, the amplicon sequence, and the oligonucleotide sequence. Here, Tm is a temperature indicating binding affinity between the nucleic acid sequence and the oligonucleotide. The Tm may mean a temperature at which 50% of a double helix structure and a single strand each are present as the double helix structure of DNA (or RNA and a complementary strand to the RNA) is dissociated into a single strand in the nucleic acid sequence to be trained. The Tm may be a temperature in a dynamic equilibrium state. The Tm varies depending on the length and base composition of the targeted sequence, and for example, the larger the Tm value, the stronger the bond between the nucleic acid sequence and the primer, which means that it is relatively poorly dissociated into a single strand. In an embodiment, the Tm may be a Tm measured through a test performed under a pre-set environment, or may be a Tm modeled using a predetermined algorithm to predict a measured Tm.

The training data according to an embodiment may further include a length of at least one of the nucleic acid sequence, the amplicon sequence, and the oligonucleotide sequence. The length according to an embodiment may be indicated as a number of counted bases included in the corresponding sequence or a number of corresponding base pairs (bp). For example, the training input data may further include a length of the nucleic acid sequence (e.g., 1000 bp), a length of the primer sequence (e.g., 100 bp), and a length of the amplicon sequence (e.g., 166 bp).

The training data according to an embodiment may further include a type of nucleic acid sequence, and may further include a GC content among bases included in at least one of the nucleic acid sequence, the amplicon sequence, and the oligonucleotide sequence. In one embodiment, the types of nucleic acid sequences may include DNA and RNA. In addition, the GC content refers to the ratio of the base pairs or the number including G or C among the bases of the corresponding sequence, and the larger the GC content, the higher the binding affinity.

The training data according to an embodiment may include a result of an amplification reaction for the corresponding nucleic acid sequence. In an embodiment, the amplification reaction result may include at least one selected from the group consisting of an amplification inhibition level and an amplification inhibition (or an amplification non-inhibition).

Here, the amplification inhibition level represents a level at which the amplification reaction is inhibited by the nth-order structure, and specifically, it may be numerical data regarding the level at which the amplification reaction for the corresponding nucleic acid sequence is inhibited by the formation of the nth-order structure in the corresponding nucleic acid sequence.

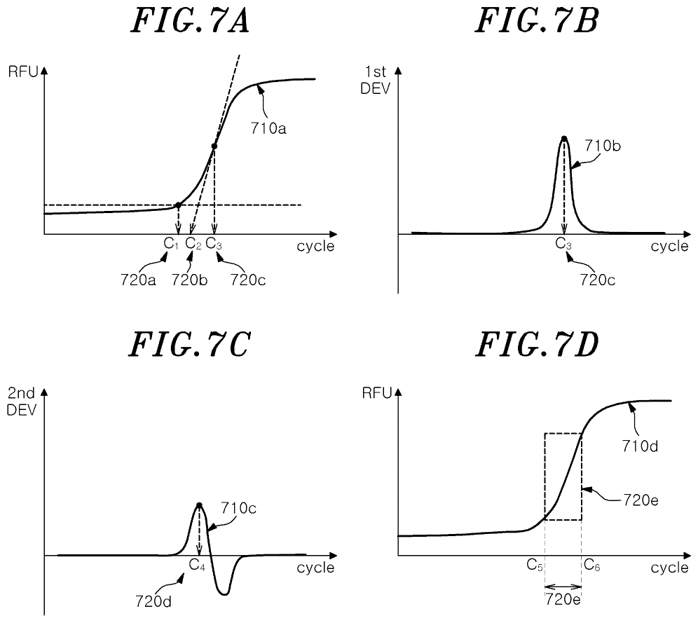

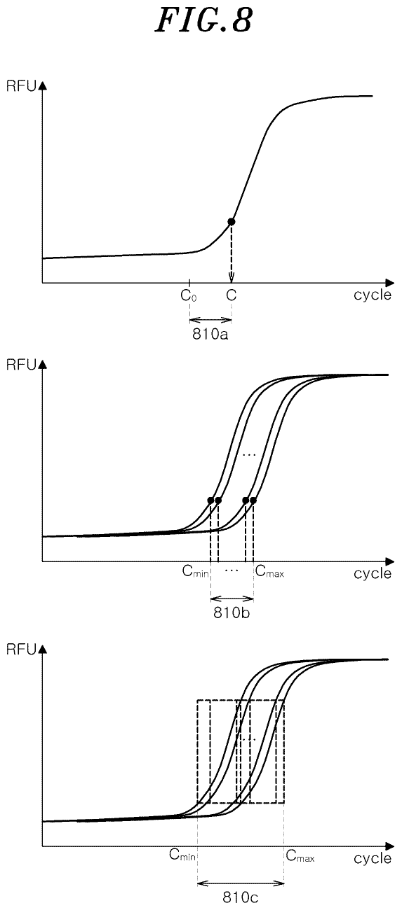

In an embodiment, the amplification inhibition level may be calculated using a cycle value corresponding to an amplification point or an amplification region in the dataset including the signal value for each cycle for the amplification reaction. Here, the amplification point refers to a specific cycle value corresponding to a reaction time or a reaction number in which the amplification of the signal value proceeds more than a predetermined level. In addition, the amplification region refers to a cycle section corresponding to a response time section or a response number section in which the amplification of the signal value proceeds more than a predetermined level, and may be understood as a concept including the amplification point according to an embodiment. In an embodiment, the cycle value corresponding to the amplification point or the amplification region may include (i) a cycle value in which a primary or secondary derivative result for the curve connecting the signal value for each cycle in the dataset is the maximum or the minimum and/or (ii) a specific cycle value in which a signal value in the dataset reaches a preset threshold value. This will be described with reference to FIGS. 7A-7D and 8.

FIGS. 7A-7D show a diagram illustrating an amplification point, an amplification region, and a specific cycle value used to calculate an amplification inhibition level according to an embodiment.

Referring to FIG. 7A, the dataset may be a set of coordinate values including a cycle(s) and a signal value(s), and may be indicated as a coordinate value(s) on a two-dimensional Cartesian coordinate system. In the coordinate system, the X-axis may indicate the cycle value, and the Y-axis may indicate the signal value (e.g., relative fluorescence units (RFUs)) measured or processed in the corresponding cycle.

In an embodiment, the specific cycle value may include a first cycle value (C₁) 720a, which is a cycle value in which a signal value in a curve 710a connecting signal values for each cycle in the dataset reaches the threshold value, and may include, for example, a cycle threshold (Ct). Here, the Ct may be interpreted to mean a cycle value indicated as a time or reaction number when the intensity of the signal value in the corresponding dataset reaches a predetermined threshold, but is not limited thereto. According to an embodiment, the Ct may be broadly interpreted as a meaning encompassing a signal value, a cycle value, or a measurement value of a specific parameter when a predetermined analysis result (e.g., a derivative of the amplification curve) derived from the corresponding dataset satisfies a predetermined condition, and may be interpreted as a meaning encompassing, for example, terms such as a cross point (CP), a take-off point (TOP), or a quantization cycle (CQ) used in the art.

In an embodiment, the amplification point may include a cycle value in contact with the maximum slope in the curve 710a connecting the signal values for each cycle. For example, as shown in FIG. 7A, the curve 710a connecting the signal values for each cycle may include a second cycle value (C₂) 720b, which is a cycle value in which a straight line according to the slope at the inflection point intersects the X-axis or a straight line according to the threshold value.

In an embodiment, the amplification point may include a cycle value in which the primary derivative result 710b or the or secondary derivative result 710c with respect to the curve 710a connecting the signal values for each cycle becomes the maximum or the minimum.

For example, referring to FIG. 7B, the primary derivative result 710b for the curve 710a connecting signal values for each cycle in the dataset may be obtained, and the amplification point may include a third cycle value (C₃) 720c in which the primary derivative result 710b satisfies a specific condition (e.g., the first derivative value is maximum). The third cycle value (C₃) 720c may include, for example, a first derivative maximum (FDM) or a slope regression-first derivative maximum (SR-FDM).

As another example, referring to FIG. 7C, the secondary derivative result 710c for the curve 710a connecting signal values for each cycle in the dataset is obtained, and the amplification point may include a fourth cycle value (C₄) 720d, which is a cycle value in which the secondary derivative result 710c becomes the maximum. The fourth cycle value (C₄) 720d may be, for example, a second derivative maximum (SDM) or a slope regression-second derivative maximum (SR-SDM).

In an embodiment, the amplification region may include a cycle section in which the signal value within the curve 710a connecting the signal values for each cycle or a slope of the signal value satisfies a predetermined condition. For example, referring to FIG. 7D, the amplification region may include an amplification cycle section 720e in which a magnitude of the signal value is between a preset first ratio (e.g., 25%) to a preset second ratio to the total signal change amount within the curve 710a connecting the signal values for each cycle, or a signal value section corresponding to the amplification cycle section 720e. The amplification point and the amplification region are not limited to the above-described embodiments, and various known analysis indicators used in the art to analyze the amplification result may be applied.

FIG. 8 is a diagram illustrating the amplification inhibition level according to an embodiment.

Referring to FIG. 8A, the amplification inhibition level according to an embodiment may be calculated based on a comparison result between the amplification point and a reference amplification point. For example, the amplification inhibition level may be a first amplification inhibition level 810a obtained by subtracting the reference amplification point (C₀) from any one of the first cycle value (C₁) 720a to the third cycle value (C₃) 720c. The reference amplification point (C₀) according to an embodiment may mean an ideal amplification region in the case where the nth-order structure is not formed or the amplification reaction is not inhibited even if the nth-order structure is formed in the amplification reaction for the corresponding nucleic acid sequence. In an embodiment, the reference amplification point may be a pre-stored set value, or may be a measured value or a statistical value determined from two or more data sets obtained for the corresponding nucleic acid sequence (this will be described later).

Referring to FIG. 8B, the amplification inhibition level according to an embodiment may be calculated based on the comparison result between the amplification points in each of a plurality of datasets. Such the plurality of datasets may mean a plurality of datasets obtained from a plurality of amplification reactions for the corresponding nucleic acid sequence, and each dataset may include signal values for each cycle. For example, curves connecting the signal values for each cycle in each of the plurality of data sets are plotted as shown in FIG. 8(b), and the third cycle value (C₃) 720c may be determined in each curve. In addition, the amplification inhibition level may be a second amplification inhibition level 810b that is a result obtained by calculating the third cycle value (C₃) 720c determined as described above in a preset calculation method. For example, the second amplification inhibition level 810b may be calculated by a method of calculating a difference between the maximum cycle value Cmax and the minimum cycle value Cmin from among a plurality of third cycle values (C₃) 720c, or a method of calculating a cycle section within a predetermined percentage from a value distribution of the plurality of third cycle values (C₃) 720c.

Referring to FIG. 8C, the amplification inhibition level according to an embodiment may be calculated based on the comparison result between the amplification region and the reference amplification region, or may be calculated based on the comparison result between the amplification regions in each of the plurality of datasets, and may be understood in a manner similar to the embodiments described above with reference to FIGS. 8A and 8B. As an example, when the amplification regions in each of the plurality of datasets is plotted as shown in FIG. 8(c), the amplification inhibition level may be a third amplification inhibition level 810c representing a result of calculating the section difference between the amplification regions or a result obtained by digitizing the pattern of the amplification change appearing in each amplification region.

A detailed method of calculating the amplification inhibition level will be described in a section on a process of obtaining the training data, which will be described later. Meanwhile, according to an embodiment, the meaning that the amplification reaction is inhibited may be broadly interpreted as a meaning that the efficiency at which the target nucleic acid is amplified in the amplification reaction is changed. For example, the amplification efficiency may be determined by using the amplification point, the amplification region, or Ct, and the amplification inhibition level may be determined from the amplification efficiency or the amount of change in the amplification efficiency.

Meanwhile, in another embodiment, the amplification reaction result may include an amplification inhibition or an amplification non-inhibition representing whether the amplification inhibition level satisfies a predetermined criterion. Here, the amplification inhibition or the amplification non-inhibition may be indicated as label data for whether the amplification is inhibited or not determined through comparison between the amplification inhibition level and a preset reference value. In an embodiment, the amplification inhibition (or the amplification non-inhibition) may be labeled with a first label (e.g., 1) indicating that the amplification is inhibited if the amplification inhibition level is not less than the reference value (e.g., 2), and may be labeled with a second label (e.g., 0) indicating that the amplification is not inhibited if the amplification inhibition level is less than the reference value.

### Process of Obtaining the Training Data

The model learning unit 110 according to an embodiment may obtain a plurality of the training data. In an embodiment, each set of the training data may include at least one of a first data group and a second data group determined from the first data group.

In an embodiment, the model learning unit 110 may obtain a plurality of first data groups, obtain a plurality of second data groups using the plurality of first data groups, and obtain a plurality of training data including at least one of the plurality of first data groups and the plurality of second data groups. In another embodiment, the model learning unit 110 may load a plurality of the training data from the memory 100 or the storage device by the processor 300, or may receive the plurality of the training data from another device through the communication unit 200. Hereinafter, the former embodiment will be described in more detail.

The model learning unit 110 may obtain the plurality of first data groups. Here, each first data group may include a predetermined nucleic acid sequence and a reaction condition used in an amplification reaction for the corresponding nucleic acid sequence. According to embodiments, the first data group may be understood as configuration data about a predetermined nucleic acid sequence prepared for generating the training data and an environment of the corresponding amplification reaction.

Here, the nucleic acid sequences and/or the reaction condition of the plurality of first data groups may be at least partially different. For example, when comparing any first data groups among the plurality of first data groups, at least a portion of the nucleic acid sequence may be different, at least a portion of the data included in the reaction condition may be different, or both the nucleic acid sequence and the reaction condition may be at least partially different.

The reaction condition may broadly mean various information required in the process of the nucleic acid amplification reaction, such as a reaction environment of the nucleic acid amplification reaction or a condition for a material put to make the reaction environment. In an embodiment, the reaction condition may include at least one of a reaction medium used for the nucleic acid amplification reaction, a nucleic acid sequence condition, an oligonucleotide condition, an environmental condition, and other conditions (e.g., temperature, pressure, and time).

Here, the reaction medium is a material surrounding the reaction environment, and in an embodiment, may include materials put into a reaction well to create the reaction environment so that one or more of the plurality of steps (e.g., step denaturation, step annealing, and step extension) for the nucleic acid amplification reaction are performed. In an embodiment, the reaction medium may be one or more materials selected from the group consisting of a pH-related substance (e.g., tris buffer, ethylene-diamine-tetraacetic acid (EDTA)) that affects pH, an ion strength-related material (e.g., ionic material, which is Mg2+, K+, Na+, NH4+, or Cl−) that affects ionic strength, an enzyme (e.g., nuclease, polymerase, ligase, or modifying enzyme) used for nucleic acid transfer or linkage in the nucleic acid amplification reaction, and an enzyme stabilization-related material (e.g., sugar) for enzyme stabilization. In addition, the nucleic acid sequence condition may include a condition for at least one of the amount (e.g., concentration) of a sample containing a nucleic acid sequence to be provided to the reaction well, a type (e.g., species) of an organism (e.g., host) having the corresponding nucleic acid sequence, and a type (e.g., species) of the organism (e.g., host) providing the corresponding sample. In addition, the oligonucleotide condition may include a condition for at least one of a configuration of one or more oligonucleotide sets including one or more oligonucleotide sequences (e.g., forward primer sequence, reverse primer sequence), and an amount (e.g., concentration) of an oligonucleotide. In addition, the environmental condition broadly refers to a condition for a surrounding environment such as a device or an experimental space that at least partially affects the nucleic acid amplification reaction, and may include, for example, a type of a device (e.g., a reaction well, a plate, a nucleic acid extraction device, an amplification device, etc.) used in the process of the nucleic acid amplification reaction. In addition, the other conditions may be temperature, pressure, and time conditions provided to the reaction well for the progress of one or more of the plurality of steps for the nucleic acid amplification reaction. The above-described embodiments are exemplary, and various types of materials, conditions, and information known in the art may be used.

As described above, each first data group may include a predetermined specific nucleic acid sequence, and the type, concentration, or size of the nucleic acid sequence in the reaction conditions required for the amplification reaction for the corresponding nucleic acid sequence may be set.

In an embodiment, the model learning unit 110 may load the plurality of first data groups from the memory 100 or the storage device by the processor 300, and in another one embodiment, may receive a user input for the nucleic acid sequence or the reaction condition in each first data group from an input/output device, and in another one embodiment, may receive the plurality of first data groups from another device through the communication unit 200.

Meanwhile, the nucleic acid sequence according to an embodiment may be data obtained from a public database or data processed, modified, or separated therefrom. For example, virus sequences of specific species are collected from the public database such as national center for biotechnology information (NCBI), global initiative for sharing all influenza data (GISAID), and/or American type culture collection (ATCC), and alignment for the collected virus sequences and a search for a conserved region from the alignment results is performed, whereby one or more nucleic acid sequences for learning may be determined. In addition, an oligonucleotide sequence (e.g., a primer sequence or a probe sequence) bound to the corresponding nucleic acid sequence may be a sequence designed from a part of bases included in the corresponding nucleic acid sequence.

The model learning unit 110 may obtain the plurality of second data groups using the plurality of first data groups. The plurality of second data groups according to an embodiment may include embodiments of the above-described training data. For example, each second data group may include the first analysis data for the nth-order structure present in the corresponding nucleic acid sequence, and may further include at least one of the nucleic acid sequence, the amplicon sequence, the oligonucleotide sequence, the Tm, the length, the type, and the GC content. In addition, each second data group may include an amplification reaction result for the corresponding nucleic acid sequence, and the amplification reaction result may include, for example, at least one of numerical data for the corresponding amplification inhibition level and label data for an amplification inhibition or an amplification non-inhibition. Hereinafter, embodiments of generating data included in each second data group by using each first data group will be described.

The model learning unit 110 may obtain the first analysis data for the nth-order structure at least partially based on the nucleic acid sequence included in each first data group and the corresponding reaction condition. For example, the model learning unit 110 may apply a predetermined nucleic acid sequence included in each first data group, a type (e.g., DNA or RNA) of the nucleic acid sequence, and a temperature (e.g., 60 degrees) to a pre-stored thermodynamic property calculation algorithm (e.g., a free energy calculation algorithm), and may calculate thermodynamic data (e.g., an amount of change in Gibbs free energy) for the formation of a specific nth-order structure (e.g., hairpin) (see identification number 30) within the entire range (see identification number 20) of the corresponding nucleic acid sequence (see identification number 10) at the corresponding temperature. According to an embodiment, various known equations may be used for the above-described thermodynamic property calculation algorithm.

In an embodiment, the model learning unit 110 may calculate thermodynamic data for the formation of an arbitrary nth-order structure using the nucleic acid sequence included in each first data group. For example, the model learning unit 110 may apply the nucleic acid sequence and the corresponding reaction condition to a free energy calculation algorithm, thereby obtaining a change in free energy in the most stable structure of the corresponding nucleic acid sequence under the corresponding reaction condition. For example, the corresponding stable structure may be classified into a case in which the nth-order structure is formed and a case in which the nth-order structure is not formed, and when the nth-order structure is not formed, the free energy change amount may be processed to 0, and information on the type of the nth-order structure may be obtained.

In one embodiment, the model learning unit 110 may calculate thermodynamic data for the formation of a specific nth-order structure using the nucleic acid sequence included in each first data group. For example, the model learning unit 110 may apply the nucleic acid sequence and the corresponding reaction condition to a free energy calculation algorithm for the formation of hairpin loop, thereby obtaining the amount of change in free energy when hairpin loop is formed in the corresponding nucleic acid sequence under the corresponding reaction condition.

In an embodiment, the model learning unit 110 may calculate thermodynamic data for the formation of each of the plurality of nth-order structures using the nucleic acid sequence included in each first data group. For example, the model learning unit 110 may apply the nucleic acid sequence and the corresponding reaction condition to a preset free energy calculation algorithm for the formation of each of hairpin loop, internal loop, bulge loop, and multi-loops. Accordingly, the model learning unit 110 may obtain each free energy change amount when each of hairpin loop, internal loop, bulge loop, multi-loops and G-quadruplex is formed in the corresponding nucleic acid sequence under the corresponding reaction condition.

In an embodiment, the model learning unit 110 may determine the first block unit 20a from the nucleic acid sequences included in each first data group, and may calculate the thermodynamic data of the first block unit 20a using the partial sequence corresponding to the first block unit 20a of the nucleic acid sequences and the reaction medium and temperature conditions of the corresponding reaction conditions. For example, the model learning unit 110 may determine a region (see FIG. 20A) within a preset distance (e.g., 10 bp) based on a region in which an oligonucleotide sequence (see identification number 40) is annealed to the corresponding nucleic acid sequence as the first block unit 20a. The model learning unit 110 may calculate the amount of change in the first Gibbs free energy (ΔG₁) by applying the partial sequence corresponding to the first block unit 20a, the reaction medium (e.g., a salt concentration based on an enzyme environment), and a first temperature to a pre-stored free energy calculation algorithm. In an embodiment, the first temperature may be a value not less than 50 degrees and not greater than 70 degrees, for example, may be set to 60 degrees based on the Tm characteristics of the primer sequence.

In an embodiment, the model learning unit 110 may determine the second block unit 20b from the nucleic acid sequences included in each first data group, and may calculate the thermodynamic data of the second block unit 20b using the partial sequence corresponding to the second block unit 20b in the nucleic acid sequences and the reaction medium and temperature conditions of the corresponding reaction conditions. For example, the model learning unit 110 may determine a region in which an oligonucleotide sequence is annealed and then extended as the second block unit 20b. The model learning unit 110 may calculate the amount of change in the second Gibbs free energy (ΔG₂) by applying the partial sequence corresponding to the second block unit 20b, the reaction medium (e.g., a salt concentration based on an enzyme environment), and a second temperature to the free energy calculation algorithm. In an embodiment, the second temperature may be a value not less than 50 degrees and not greater than 70 degrees, for example, may be set to 60 degrees based on amplicon characteristics generated as a product in the process of the amplification reaction.

In an embodiment, the model learning unit 110 may determine the third block unit 20c from the nucleic acid sequences included in each first data group, and may calculate the thermodynamic data of the third block unit 20c using the partial sequence corresponding to the third block unit 20c in the nucleic acid sequences and the reaction medium and temperature conditions of the corresponding reaction conditions. For example, the model learning unit 110 may determine the entire range (see the identification number 20c) of the nucleic acid sequence (see the identification number 10) as the third block unit 20c. The model learning unit 110 may calculate the amount of change in the third Gibbs free energy (ΔG₃) by applying the entire sequence corresponding to the third block unit 20c, the reaction medium (e.g., salt concentration based on the enzyme environment), and the third temperature to the free energy calculation algorithm. In an embodiment, the third temperature may be a value not less than 40 degrees and not greater than 70 degrees, for example, may be set based on a reverse transcription step and/or an annealing step according to the type of nucleic acid sequence (e.g., DNA or RNA). For example, in the case of RNA, the third temperature may be set to 50 degrees in consideration of the temperature of the initial reverse transcription step during the amplification reaction. In the case of DNA, the third temperature may be set to 60 degrees in consideration of the temperature of the annealing step in which the reverse transcription step is omitted.

In an embodiment, at least one of an MFE calculation equation (e.g., NUPACK algorithm) for calculating a minimum free energy (MFE) representing thermodynamic energy in the most stable state among all structures that a molecule may have, an entropy calculation equation and an entropy calculation equation, and the like may be applied to the free energy calculation algorithm, and for example, NUPACK algorithm, dynamic programming algorithm, energy combining data, and the like may be used. For example, by applying data of a base sequence in which types of bases included in a nucleic acid sequence are sequentially listed, and reaction conditions with respect to a temperature and an ionic material to an MFE calculation algorithm, an amount of change in Gibbs free energy (ΔG) for a secondary structure formed in the nucleic acid sequence may be calculated under the corresponding temperature and concentration conditions of the ionic material. According to an embodiment, a process of correcting by applying a salt concentration in the equation after calculating the MFE for the partial sequence of the above-described block unit may be added. The salt concentration is an index indicating the thermodynamic stability of the double strand of the nucleic acid sequence and may be determined from the reaction medium (e.g., concentration of an ionic material, enzyme, etc.). As the salt concentration is applied to the MFE, information on the experimental environment in which the amplification reaction is performed may be reflected in the calculation of free energy. This MFE calculation method has the advantage of improving the prediction accuracy of the molecular structure by reducing the number of structures that the molecule can take. However, the above-described free energy calculation algorithm is not limited to the above-described embodiments, and various known forms of free energy calculation equations may be applied.

The model learning unit 110 may obtain the analysis data for the structural characteristics of a specific nth-order structure appearing at bases in the nucleic acid sequence, based at least in part on the nucleic acid sequence included in each first data group.

In an embodiment, model learning unit 110 may apply the nucleic acid sequence to a previously stored G-quadruplex score calculation algorithm (e.g. G4 hunter algorithm), may calculate the G-quadruplex score (e.g., G4 hunter value) that quantifies G-richness and/or G-skewness based on the position, number, and/or distribution of G in the corresponding nucleic acid sequence, and may obtain the analysis data for the structural characteristics of the G-quadruplex including the G-quadruplex score. As an example, the model learning unit 110 may calculate the G4 hunter value by calculating the overall score in a method of applying a set score for each base to each base in the nucleic acid sequence and assigning weight to consecutive G or C. In addition, the model learning unit 110 may calculate the G4 hunter value for each partial sequence by using a method of shifting a partial sequence corresponding to a preset range (e.g., 25 mer) within the nucleic acid sequence by a preset unit (e.g., 1 mer), and may calculate a G4 hunter representative value by performing statistical calculations (e.g. average value, maximum value, mode value, etc.) on the calculated G4 hunter values.

In an embodiment, the model learning unit 110 may obtain the analysis data for the structural characteristics of the G-quadruplex by using at least one of the nucleic acid sequence, the amplicon sequence, and the oligonucleotide sequence. For example, the G-quadruplex score may include (a) an average and/or maximum value of the G4 hunter values calculated in the above manner for the nucleic acid sequence, and (b) the average and/or maximum value of the G4 hunter values calculated in the above manner for the amplicon sequence.

In an embodiment, the model learning unit 110 may obtain the analysis data for the structural characteristics of any one of the hairpin loop, the internal loop, the bulge loop, the multi-loop, the pseudoknot, the kissing hairpin, and the hairpin-bulge contact. For example, the model learning unit 110 may quantify the degree to which the number or aspect of a specific base combination (e.g., the number of patterns in which consecutive G is more than a predetermined number, the maximum consecutive length of G allowing up to a certain number of mismatches) that is mainly seen for each specific type of the nth-order structure appears within the corresponding base sequence. The model learning unit 110 may calculate the characteristic score that better represents the structural characteristics of the nth-order structure of the corresponding type by applying the quantified degree to a previously stored mathematical equation.

The model learning unit 110 may obtain the Tm of at least one of the nucleic acid sequence, the amplicon sequence, and the oligonucleotide sequence based at least in part on the nucleic acid sequence included in each first data group and the corresponding reaction condition. In an embodiment, the model learning unit 110 may calculate a prediction value for the Tm of the oligonucleotide based on the binding state of the nucleic acid sequence and the oligonucleotide sequence by applying the nucleic acid sequence and the oligonucleotide sequence (e.g., a forward primer sequence, a reverse primer sequence) to a pre-stored Tm prediction algorithm. The Tm prediction algorithm is an algorithm for predicting a Tm actually measured under the corresponding reaction condition, and may provide a modeled Tm value by at least partially applying the corresponding reaction condition, and for example, Biopython algorithm or the like may be used. In another embodiment, the model learning unit 110 may derive an actual measurement value for Tm from a test result for Tm of an oligonucleotide performed under the corresponding reaction condition. For example, a melting curve analysis (MCA) analysis method may be used for the test.

The model learning unit 110 may calculate the GC content using at least one of the nucleic acid sequence, the amplicon sequence, and the oligonucleotide sequence included in each first data group. For example, the model learning unit 110 may calculate GC content by counting a ratio of the number of G and C among A, G, C, and T, which are all bases included in each of the nucleic acid sequence, the amplicon sequence, and the oligonucleotide sequence.

The model learning unit 110 may calculate the length of at least one of the nucleic acid sequence, the amplicon sequence, and the oligonucleotide sequence using the nucleic acid sequence and the oligonucleotide sequence included in each first data group. For example, the model learning unit 110 may count base pairs for each of the nucleic acid sequence and the oligonucleotide sequence (e.g., forward primer sequence, reverse primer sequence) to calculate the length (e.g., 1000 bp) of the nucleic acid sequence and the length (e.g., 100 bp) of the oligonucleotide sequence, and may calculate the length (e.g., 166 bp) of the amplicon sequence derived from annealing and extension of the oligonucleotide sequence for the corresponding nucleic acid sequence.

The model learning unit 110 may generate each set of training data comprising at least one of each first data group and each second data group. For example, each set of the training data may include a second data group. In addition, each set of the training data may further include at least one at least one data which is included in each first data group and does not require the calculation process, for example, the nucleic acid sequence, the type of the nucleic acid sequence (e.g., DNA or RNA), and each data of reaction conditions (e.g., an identification number of a reaction medium set, an identification number of a reaction condition set, a reaction temperature, etc.).

The model learning unit 110 may generate at least one of the amplification inhibition level, and the amplification inhibition or the amplification non-inhibition based at least in part on the nucleic acid sequence included in each first data group and the reaction condition. Specifically, the model learning unit 110 may obtain a plurality of datasets from a plurality of amplification reactions for the corresponding nucleic acid sequence, and determine the amplification inhibition level and/or the amplification inhibition (or the amplification non-inhibition) from the obtained plurality of datasets.

In an embodiment, for each of the plurality of first data groups, the model learning unit 110 may obtain two or more datasets from two or more amplification reactions for the nucleic acid sequence performed under the reaction condition included in each of the first data groups. For example, regarding the data group in which a first reaction condition is set with a first nucleic acid sequence, an amplification reaction for the first nucleic acid sequence may be performed under the same first reaction condition, thereby obtaining m₁ (e.g., 96) datasets for the first nucleic acid sequence. In another example, regarding the data group in which a second reaction condition is set with a second nucleic acid sequence, an amplification reaction for the second nucleic acid sequence may be performed under the same second reaction condition, thereby obtaining m₂ (e.g., 48) datasets for the second nucleic acid sequence. According to an embodiment, the same reaction conditions may mean that the values of the types, concentrations, or sizes of various element conditions (e.g., reaction medium, temperature, etc.) included in the above-described reaction conditions are the same, or substantially the same as each other since the values are within a predetermined range.

Meanwhile, when an amplification reaction is performed using a plate in which a plurality of reaction wells are accommodated, a plurality of amplification reactions for one identical nucleic acid sequence may be performed together to obtain a plurality of datasets, and a number of the plurality of datasets (e.g., m₁ and m₂) may be the same or different according to each reaction condition. In addition, two or more datasets for each nucleic acid sequence may be acquired together when the corresponding first data group is obtained, or may be obtained by performing an amplification reaction under the corresponding reaction conditions after the first data group is obtained. For example, as described above, after the first data group is prepared first, the first data group and the datasets may be sequentially obtained in such a manner that an amplification reaction is performed under the corresponding reaction condition. For another example, after two or more datasets performed under a predetermined reaction condition are obtained for a predetermined nucleic acid sequence, when experimental data including the corresponding nucleic acid sequence, reaction condition, and datasets are obtained, the nucleic acid sequence and reaction condition of the first data group may be derived from the experimental data. For another example, a plurality of datasets, which are performed under a plurality of reaction conditions on a plurality of nucleic acid sequences, may be obtained first. And then, datasets, which are performed under the same reaction conditions on the same nucleic acid sequence, may be filtered from the plurality of datasets. Alternatively, the first data group and the datasets may be prepared and obtained together.

In an embodiment, the two or more datasets may be processed to exclude an influence of one or more preset exclusion variables different from the nth-order structure. For example, the model learning unit 110 may output a signal requesting that the two or more datasets are to be performed under the above-described same reaction condition. When the two or more datasets are obtained, the model learning unit 110 may analyze whether the influence of the exclusion variable is included in the two or more datasets. In addition, when it is analyzed that the influence of the exclusion variable is included in the two or more datasets, the model learning unit 110 may exclude the two or more datasets so that they are not used as training data. Alternatively, the model learning unit 110 may obtain the remaining datasets by excluding one or more datasets including the influence of the exclusion variable from the two or more datasets, and may control so that the remaining datasets are used as training data.

In an embodiment, the exclusion variable may include a dimer variable. As a specific example, for 95 amplification reactions for a predetermined nucleic acid sequence, an amplification reaction may be performed using one plate in which 96 reaction wells are accommodated, 95 of the 96 reaction wells contain a first sample including an target analyte corresponding to the nucleic acid sequence, the remaining one contains a second sample not including the target analyte corresponding to the nucleic acid, and under the above-described reaction conditions, the amplification reaction for the plate may be performed together. In this case, a process of confirming the influence due to the dimer variable as one of the exclusion variables through the reaction wells containing the second sample may be performed. Illustratively, when amplification occurs even though the target analyte corresponding to the nucleic acid sequence is not included in the second sample, it may be estimated that a self-dimer occurs inside an oligonucleotide (e.g., a primer), or a pair-dimer occurs between oligonucleotides (e.g., between a forward primer and a reverse primer, between forward primers, or between reverse primers). In this case, it is determined that the 95 datasets obtained from the 95 reaction wells are affected by the dimer variable and the datasets may be excluded in the process of generating the training data.

In another embodiment, the exclusion variable may further include a variable related to at least one of the Tm of the oligonucleotide set, the number of oligonucleotides, the concentration, the temperature, and the number of target analytes. The influence of the variable may be confirmed by adding a reaction well for confirming the control of the variable in the plate as described above, or by various known methods for confirming whether the variable is controlled in the process of the amplification reaction.

In this way, data sets that are analyzed as having an effect due to the exclusion variable and determined to be unsuitable may be removed from sample datasets used to generate the training data. The removal is a significant operation in measuring the amplification inhibition level by the nth-order structure, and has an advantage of increasing the reliability of the amplification inhibition level.

In this regard, as described above, the thermodynamic data (e.g., the amount of change in Gibbs free energy) of the nth-order structure represents the thermodynamic stability of the nth-order structure, and the high thermodynamic stability may mean that the probability of the nth-order structure being formed in the nucleic acid sequence is relatively high. However, in the related art, there is a limitation in that the correlation between whether the amplification reaction is substantially inhibited or to what extent the amplification reaction is inhibited is not known.

However, when a plurality of amplification reactions are performed on a specific nucleic acid sequence under the same predetermined reaction conditions, the influence of most variables on a plurality of datasets obtained from the plurality of amplification reactions can be controlled. Accordingly, in the plurality of datasets, as variables other than the influence of the nth-order structure are controlled, relatively identical amplification results are shown, but the formation of the secondary structure in the corresponding nucleic acid sequence is stochastically shown, and thus the difference in the affected amplification results may also stochastically be shown. Accordingly, as described above, when the datasets are processed to exclude the influence of the exclusion variable, the influence of the variables other than the nth-order structure is minimized in the datasets used for training data, and the influence due to the formation of the nth-order structure is included, which may appear as a difference in the amplification results such as the amplification point or the amplification region. As a result, by measuring the difference in the amplification results, it is possible to effectively quantify the degree to which the amplification reaction for the corresponding nucleic acid sequence is affected by the secondary structure.

The model learning unit 110 may calculate the amplification inhibition level due to the nth-order structure of the corresponding nucleic acid sequence from two or more datasets for each nucleic acid sequence. Specifically, the above-described two or more datasets obtained for the predetermined nucleic acid sequence may include at least one of a normal amplification case in which amplification is well performed because a secondary structure is not formed in the nucleic acid sequence, an abnormal amplification case at a level in which amplification is not substantially inhibited even if a secondary structure is formed, and an amplification inhibition case at a level in which amplification is inhibited due to a secondary structure formed in the nucleic acid sequence. The model learning unit 110 may calculate the amplification inhibition level by measuring a difference between the amplification point or the amplification region between the normal amplification case and the amplification inhibition case appearing in the two or more data sets obtained as described above. Through the amplification inhibition level, it is possible to quantify the level at which amplification is inhibited by the secondary structure compared to normal amplification for each nucleic acid sequence.

In an embodiment, the amplification inhibition level may be calculated by using the difference between the amplification point, the difference in the amplification region, and/or the difference in the specific cycle value in the two or more datasets described above.

For example, as shown in FIG. 8B, the model learning unit 110 may measure each amplification point described above (e.g., the third cycle value C₃) from each of m₁ curves obtained by connecting data points in each of m₁ (e.g., 96) datasets of the first nucleic acid sequence. In addition, the model learning unit 110 may calculate the amplification inhibition level (e.g., the second amplification inhibition level 810b) for a level at which the amplification reaction for the first nucleic acid sequence is inhibited by the nth structure in the first nucleic acid sequence, by using the comparison result between the values of the measured m₁ amplification points. In an embodiment, as described above, the method of obtaining the comparison result may include a method of obtaining a difference (e.g., Cmax-Cmin) between the m₁ amplification point values, a method of performing arithmetic calculation on at least some of the m₁ amplification point values according to a preset equation (e.g., (Cmax−Cmin)*weight, etc.), and a method of performing statistical calculation on one or more representative values (e.g., an average, a median, a mode, a specific quartile (e.g., a first quartile, a third quartile, etc.) or a representative range (e.g., an interquartile range (IQR), etc.)) for the m₁ values by using a pre-stored statistical algorithm (e.g., box plot).

As another example, as shown in FIG. 8A, the model learning unit 110 may measure each amplification point described above (e.g., the second cycle value C₂ (720b)) from each of m₂ curves connecting data points in each of m₂ (e.g., 48) datasets of the second nucleic acid sequence. In addition, the model learning unit 110 may calculate the amplification inhibition level (e.g., the first amplification inhibition level 810a) for a level at which the amplification reaction for the second nucleic acid sequence is inhibited by the nth-order structure in the second nucleic acid sequence, by using the comparison result between the reference amplification point C₀ and the representative amplification point C determined from the measured m₂ amplification point values. Illustratively, the reference amplification point C₀ may be the lowest value among the m₂ values, and the representative amplification point C may be a representative value (e.g., a third quartile, maximum value) statistically calculated from the measured m₂ values.

As another example, as shown in FIG. 8C, the model learning unit 110 may measure each amplification region described above (e.g., the amplification cycle section 720e) from each of m₃ curves obtained by connecting data points in each of m₃ (e.g., 96) datasets of the third nucleic acid sequence. In addition, the model learning unit 110 may calculate the amplification inhibition level (e.g., the third amplification inhibition level 810c) for a level at which the amplification reaction for the third nucleic acid sequence is inhibited by the nth-order structure in the third nucleic acid sequence, by using the comparison result between the m₃ amplification regions. In an embodiment, as described above, the method of obtaining the comparison result may include a method of obtaining a section difference between a minimum cycle and a maximum cycle in a graph where the m₃ amplification regions overlap, a method of obtaining a difference between aspects (e.g., a gradient of an amplification curve) of the amplification change each appearing in each amplification region, and the like. Meanwhile, for convenience of explanation, the embodiment of the amplification region and the embodiment of the amplification point are separately described in the specification, but the amplification region according to the embodiment of the present disclosure may be broadly interpreted as including such an amplification point.

The model learning unit 110 may determine the amplification inhibition or the amplification non-inhibition according to whether the amplification inhibition level satisfies a predetermined criterion. In an embodiment, the predetermined criterion may include at least one of a condition for comparing the magnitude of the amplification inhibition level with the magnitude of the reference value and a condition for determining whether the magnitude of the amplification inhibition level is within one of a plurality of value ranges. For example, when the calculated amplification inhibition level is not less than the reference value (e.g., 2), the model learning unit 110 may determine the label of the amplification inhibition as a first label (e.g., 1), and when the calculated amplification inhibition level is not greater than the reference value, may determine the amplification inhibition level as a second label (e.g., 0). As another example, a plurality of intervals corresponding to a plurality of amplification inhibition intensities (e.g., strong inhibition, medium inhibition, weak inhibition, and no inhibition) are preset. In addition, according to the amplification inhibition intensity corresponding to the interval to which the amplification inhibition level belongs, the model learning unit 110 may determine the label of the amplification inhibition as a label corresponding to the amplification inhibition intensity among a third label to a sixth label (e.g., 4, 3, 2, 1).

In an embodiment, the given criterion may be determined based at least in part on the value of n in the nth-order structure and/or the type of the nth-order structure in the nucleic acid sequence. As described above, the type of the nth-order structure may include at least one selected from the group consisting of the hairpin loop, the internal loop, the bulge loop, the multi-loops, the G-quadruplex and the combination thereof, when n is 2, and may include any one selected from the group consisting of the pseudoknots, the kissing hairpin, the hairpin-bulge contact, and the combination thereof, when n is 3. For example, among a plurality of pre-stored reference values or value ranges, a reference value or a value range corresponding to the value of n or the type of the nth-order structure may be applied.

The model learning unit 110 may generate the training data including the training input data and the training answer data labeled on the corresponding training input data as one training data set from at least one of each first data group and each second data group. Illustratively, the R-th training data (R is an integer not less than 2) may include the first analysis data for the nth-order structure, the R-th training input data including the Tm and the GC content, and the R-th training answer data including numerical data of the corresponding amplification inhibition level.

Table 1 below illustrates the first training data to the R-th training data according to an embodiment. Referring to Table 1, each training input data may include the type of the nucleic acid sequence (ex. templet type), the amount of change in Gibbs free energy variation (ΔG_{1A}), the amount of change in Gibbs free energy (ΔG_{1B}), the amount of change in Gibbs free energy (ΔG₂), the amount of change in Gibbs free energy (ΔG₃), the length (L_{1A}) of the block units (20a) considering the forward oligonucleotide sequence, the length (L_{1B}) of the block units (20a) considering the reverse oligonucleotide sequence, the length (L2) of the second block units (20b), and the length (L₃) of the third block units (20c). In addition, each training answer data may include numerical data (e.g., 2.54) for the amplification inhibition level (e.g., when the prediction model is the regression analysis model) or label data (e.g., the amplification inhibited: 1, the amplification not inhibited: 0) for the amplification inhibition level (e.g., when the prediction model is the classification model).

TABLE 1

| Set | 1 | 2 | . . . | R-2 | R-1 | R |
|---|---|---|---|---|---|---|
| Target | Cronobacter | Cronobacter | . . . | Norovirus G1 | Adenovirus | Adenovirus |
| | sakazakii | sakazakii | | | | |
| Template type | DNA | DNA | . . . | RNA | RNA | RNA |
| ΔG_{1A} | −2.79571 | −2.54424 | . . . | −10.0798 | −1.96863 | −1.96863 |
| ΔG_{1B} | −12.0613 | −0.58408 | . . . | −5.17531 | −9.17018 | −6.40475 |
| ΔG₂ | −11.2373 | −1.16004 | . . . | −13.2904 | −3.81555 | −23.2671 |
| ΔG₃ | −41.2969 | −16.4736 | . . . | −222.773 | −194.413 | −194.413 |
| L_{1A} | 100 | 95 | . . . | 100 | 100 | 100 |
| L_{1B} | 100 | 100 | . . . | 100 | 100 | 100 |
| L₂ | 166 | 188 | . . . | 163 | 121 | 251 |
| L₃ | 1000 | 1000 | . . . | 1000 | 892 | 892 |
| Amplification | 2.54 | 0.66 | . . . | 10.19 | 8.86 | 30.13 |
| inhibition level | | | | | | |
| Lable of the | 1 | 0 | . . . | 1 | 1 | 1 |
| amplification | | | | | | |
| inhibition | | | | | | |

Meanwhile, the above-described training data may be preprocessed in a form suitable for calculation in the prediction model. For example, such a preprocessing process may include a process of converting the expression method of the above-described data into a numerical value or vector form that may be computed in a machine learning model or a deep training model. For example, in case that the nucleic acid sequence is used as input data for the prediction model, the pre-processing process including vectorization of the nucleic acid sequence (e.g., embedding, encoding, tokenization, etc.) may be performed for computing by the prediction model. In such pre-processing, various conventional pre-processing techniques previously known (e.g., text conversion, label conversion, vector conversion, etc.) for pre-processing of a text or image may be used together. According to an embodiment, the process of obtaining the first and second data groups or the process of generating the training data by using at least one of the first and second data groups may be understood as a part of the preprocessing process for learning.

### Learning Process of the Prediction Model

The model learning unit 110 may perform a learning process of a prediction model using the plurality of training data. As a specific example, the model learning unit 110 may train a machine learning model using the plurality of training data described above, and may obtain a trained prediction model as a training result. The prediction model may be learned to provide a prediction result of an amplification reaction affected by the nth-order structure in an amplification reaction for a predetermined nucleic acid sequence when the analysis data for the nucleic acid sequence or the nth-order structure in the nucleic acid sequence is provided.

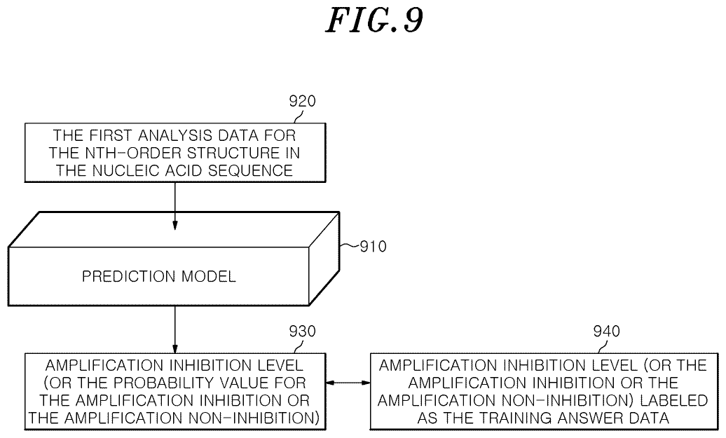

FIG. 9 exemplarily illustrates a conceptual diagram of a learning process of a prediction model 910 according to an embodiment. Here, the prediction model 910 may correspond to the prediction model before the learning is completed.

Referring to FIG. 9, the prediction model 910 may be trained using the training input data including the first analysis data 920 for the nth-order structure present in a predetermined nucleic acid sequence, such that the prediction model 910 outputs the amplification inhibition level (or a probability value for the amplification inhibition or the amplification non-inhibition) 930 by the nth-order structure of the corresponding nucleic acid sequence.

For example, in a learning process, the prediction model 910 may apply ΔG₁, ΔG₂, ΔG₃, the G-quadruplex score, Tm and GC content and the like included in the training input data to corresponding independent variables of the prediction model 1010. Accordingly, the prediction model 910 may output the amplification inhibition level (or the probability value of the amplification inhibition or the amplification non-inhibition) 930 as an output of a dependent variable. In addition, the amplification inhibition level (or the amplification inhibition or the amplification non-inhibition) 940 labeled as the training answer data in the corresponding training input data may be an expected output of the dependent variable. The prediction model 910 may be learned by using a supervised learning manner of updating the operation method of the prediction model (e.g., inversion of the coefficient value included in the predictive function) such that the error between the output of the dependent variable and the expected output is minimized.

As an embodiment, the prediction model 910 may be implemented as the regression analysis model (e.g., a Ridge linear regression analysis model, a random forest regression analysis model), and the output of the dependent variable and the expected output in the prediction model 910 may be processed in the form of the amplification inhibition level. As another embodiment, the prediction model 910 may be implemented as the classification model (e.g., a logistic regression model, a random forest classification model), and the output of the dependent variable and the expected output in the prediction model 910 may be processed in the form of the probability value the amplification inhibition (or the amplification non-inhibition) and the amplification inhibition (or the amplification non-inhibition), respectively. As another embodiment, the prediction model 910 may be implemented as a DNN having a fully connected neural network structure, and for example, a method of converting the training input data pre-processed in a vector form into a one-dimensional array and then training with the fully connected multi layered neural network may be applied. In this case, for example, the output of the prediction model 910 and the expected output may be processed in the form of the probability value for the amplification inhibition (or the amplification non-inhibition) and the amplification inhibition (or the amplification non-inhibition), respectively. The values of parameters (e.g., weights and biases) included in the artificial neural network of the prediction model 910 may be updated by a back propagation method for reducing an error between the output and the expected output.

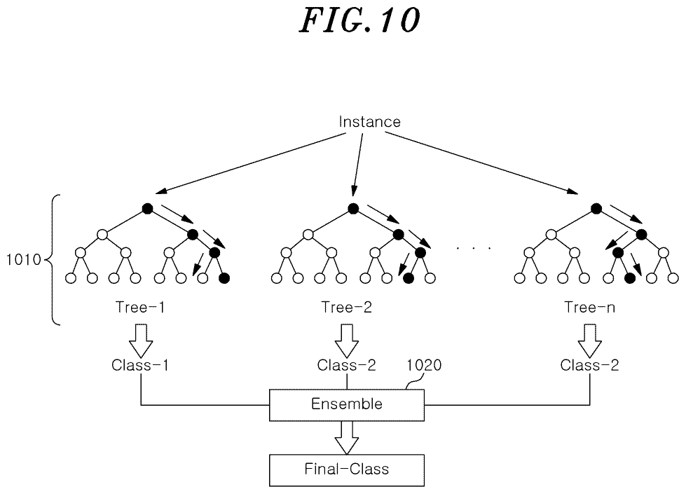

FIG. 10 is a conceptual diagram for describing a structure and an operation of a random forest-based prediction model 910 according to a first embodiment.

Referring to FIG. 10, the prediction model 910 according to the first embodiment may be implemented using the random forest model. The random forest-based prediction model 910 may include a plurality of different trees 1010 and an ensemble unit 1020.

In the first embodiment, each of the plurality of trees 1010 is a decision tree, includes a plurality of nodes (e.g., a decision node, an opportunity node, and an end node), and classification at each node may be performed based on one or more variables (e.g., ΔG₃) and classification conditions (e.g., ΔG₃≤0.948) that are classification criteria set at each node. The training input data input to the prediction model 910 may be provided to at least some of the plurality of trees 1010 as a instance for training, and in each tree 1010, training may be performed in a direction of outputting a decision on a corresponding input while passing through internal nodes and reducing entropy calculated when a classification task is performed in each node. According to an embodiment, data for training may be differently applied to each tree 1010 according to the internally performed random sampling, and each tree may be implemented to function as a different sub-model.

According to the first embodiment, the ensemble unit 1020 may provide an ensemble result by performing an ensemble of a decision output from each of the plurality of trees 1010. Although a classification model is exemplarily illustrated in FIG. 10, the random forest model according to an embodiment may be implemented as the classification model or the regression analysis model, and the ensemble unit 1020 may generate the ensemble result by performing the ensemble of a class output as a classification result or a numerical value output as a result value in each tree 1010.

For example, when the prediction model 910 is implemented as the random forest classification model, the ensemble unit 1020 may generate the ensemble result by applying a predetermined ensemble technique to the class output as the classification result in each tree 1010. As an example, when the prediction result that is the output of the prediction model 910 is categorical data for the amplification inhibition (or the amplification non-inhibition), each tree 1010 may output the class for the amplification inhibition (or the amplification non-inhibition) as the classification result, and the ensemble unit 1020 may generate a final class by applying the predetermined ensemble technique to the class output from each tree 1010. For example, the ensemble technique may include a majority voting technique for selecting a class that occupies the largest number or majority.

As another example, when the prediction model 910 is implemented as the random forest regression analysis model, the ensemble unit 1020 may generate the ensemble result by applying the predetermined ensemble technique to the amplification inhibition level or the probability of the amplification inhibition (or the amplification non-inhibition) output as a result value from each tree 1010. As an example, when the prediction result that is the output of the prediction model 910 is continuous data as the amplification inhibition level, each tree 1010 may output a numerical value of the amplification inhibition level as a result value, and the ensemble unit 1020 may calculate a final result value by applying the ensemble technique to the numerical value output from each tree 1010. For example, the ensemble technique may include a technique of calculating a representative value (e.g., an average value, a mode value, a median value, etc.) by performing a statistical operation on result values, the above-described majority voting technique, and the like.

As described above, the random forest-based prediction model 910 according to the first exemplary embodiment has advantages in terms of stable prediction performance and generalization, and has an advantage in that classification accuracy may be improved compared to the decision tree model.

The prediction model 910 according to a second embodiment may be implemented using a multiple linear regression analysis model. In an embodiment, the multiple linear regression analysis-based prediction model 910 may include a prediction function indicating a correlation between the plurality of feature included in the training input data and the amplification inhibition level by the nth-order structure. In an embodiment, the multiple linear regression analysis-based prediction model 910 may be trained based on Equations 1 to 2. For example, the prediction function may be determined based on Equation 1, and training for the prediction function may be performed based on Equation 2.

$${y}^{′} = {{\sum }_{i = 1}}^{p} ( w [ i ] \times x [ i ] + b ) \qquad EQUATION 1$$

$$MSE = \frac{1}{r} {{\sum }_{i = 1}}^{r} {( {y}_{i} + {y}_{i}^{′} )}^{2} \qquad EQUATION 2$$

Here, x₁, x₂, . . . , xₚ are independent variables of the prediction function, and correspond to each of a plurality of feature included in the training input data, and may correspond to each of P feature (P is an integer of not less than 2), for example, the thermodynamic data for the nth-order structure (e.g., ΔG_{1A}, ΔG_{1B}, ΔG₂, ΔG₃), the type of the nucleic acid sequence, the length of the nucleic acid sequence or the oligonucleotide (e.g., L_{1A}, L_{1B}, L₂, L₃), etc. In addition, w₁, w₂, . . . , wₚ represent weights for independent variables. In addition, y′ is a dependent variable of the prediction function, and means a prediction output calculated by the above-described independent variables. In addition, y corresponds to the amplification inhibition level included in the training answer data, and means the expected output of the dependent variable labeled for the corresponding training input data.

Referring to Equations 1 and 2, for example, the multiple linear regression analysis-based prediction model 910 may be trained by deriving values of a weight w and a bias b that minimize an error (e.g., a mean squared error (MSE)) between the expected output y and the predicted output y′ of the prediction function in a learning process using R sets of training data.

According to a third embodiment, the prediction model 910 may be implemented using a Lasso linear regression analysis model or a Ridge linear regression analysis model. The prediction model 910 according to the third embodiment may be trained by using a prediction function in a similar manner to the second embodiment, and may be trained by applying additional a penalty condition, together with a basic condition for finding a weight w and a bias b that minimize an error (e.g., MSE). For example, in the case of the Lasso linear regression analysis model, the L-norm regularization scheme may be applied to such additional penalty condition, and as the condition that the sum of the absolute values of the weights is minimized is applied, the training may be performed so that all elements of the weights become 0 or close to 0, and thus, no features may be used. This method has an advantage of being easy to find a generalized model through the penalty condition and easy to interpret major characteristics, such as excluding characteristics that are less necessary from the model. As another example, in the case of the ridge linear regression analysis model, the L₂-norm regularization technique may be applied to such additional penalty condition, and the training may be performed such that weights are close to 0 but do not become 0, and thus all features may be used. This method has an advantage of implementing higher prediction performance when the importance of the features is similar as a whole.

The prediction model 910 according to the second to fourth exemplary embodiments has an advantage in that it is easy to interpret the result of the model because it directly shows the effect of each characteristic of the regression coefficient on the result. Meanwhile, the types of machine learning models used for the prediction model 910 have been exemplarily described through the first to fourth embodiments, but the prediction model in the present disclosure is not limited thereto, and various types of artificial intelligence models known as described above may be applied. In another implementation, the prediction model 910 may include a Long Short Term Memory (LSTM) network, Bidirectional Encoder Representations from Transformers (BERT), or a Generative Pre-trained Transformer (GPT), etc. For example, the prediction model 910 may be learned to match the training answer data based on an input pre-processed (e.g., vector transformation) from the above-described input data for training. Alternatively, the prediction model 910 may be learned to predict an amplification reaction result by comprehensively analyzing (i) an output of a machine learning model that receives some (e.g., thermodynamic data for the nth-order structure, Tm, the length of the nucleic acid sequence, etc.) of the training input data and calculates a result, and (ii) an output of a deep training model that receives the remaining (e.g., a nucleic acid sequence) of the training input data and calculates a result.

Meanwhile, in the above-described learning process, a plurality of hyper parameters may be used. The hyper parameter may be a variable that varies by the user, and may vary depending on the type of the prediction model 910. For example, in the case where the prediction model 910 is the ridge linear regression analysis model, the hyper parameter may include a alpha (e.g., a regulation of the model), a solver type (e.g., gradient descent), or the like. In the case of the random forest regression model, the hyper parameter may include a maximum depth value (e.g., a depth of a decision tree), the number of feature (e.g., the number of feature for classification), a minimum samples_leaf value (e.g., a minimum size of data to be put in a leaf node), a minimum samples_split value (e.g., a minimum size of data to be put in an internal node), or the like. As another example, in the case of the DNN model, the hyper-parameter may include a learning rate, a cost function, the number of learning cycle iterations, weight initialization (e.g., setting a range of weight values to be weight initialization), the number of Hidden Unit (e.g., the number of hidden layers, the number of nodes of a hidden layer), and the like.

According to an embodiment of the present disclosure, in order to apply an optimized model type to the prediction model 910 or derive an optimized hyper parameter, a method of dividing the plurality of training data, performing the learning under different conditions, and then using a result of evaluating the learning may be used.

In an embodiment, the plurality of training data may be grouped into r groups (r is an integer of 2 or more). Here, each group represents a data set group for training including two or more training data sets, which are arranged in units of training data set in which the training input data and the training answer data are paired. For example, when the total number of the training data sets is 1,000, 200 data sets may be divided into five groups. In addition, the prediction model may be learned by using some of the r groups, and performance verification may be performed on the prediction model using the remaining groups. For example, the training data sets may be divided into five groups, and in the remaining four groups except for one group, the learning may be done with the training data sets of each group by applying different values of hyperparameters to each prediction model. Accordingly, the four different exemplary prediction models may be generated as targets of the performance verification, and the performance evaluation may be performed on the four different exemplary prediction models by using the training data of the corresponding one group.

In an embodiment, the values of the hyper parameters applied to the learning of the prediction model 910 may be updated based on the result of the performance verification. For example, by comparing the results of performance evaluation on the four different exemplary prediction models described above, any one exemplary prediction model having the best evaluation score may be selected, and the values of the hyper parameters applied to the selected exemplary prediction model may be determined to be used in the process of fine-tuning.

In an embodiment, in the process of determining the values of the hyper parameters, a K-fold cross validation technique may be used. For example, a method of dividing the remaining training sets except the test set among the entire training data sets that can be used for the prediction model 910 into K equal parts (e.g., K=5, 10), 1/K of which is used as a validation set, and (K−1)/K of which is used as a training set, and repeating this for each divided data set a total of K times may be used. Through this, K models may be created, and the MSE value of the prediction model may be determined according to a result of averaging mean squared error (MSE) values of each model.

In an embodiment, in the process of determining the type of the prediction model 910 and determining at least one of the hyper-parameter values, a Nested Cross Validation technique may be used. For example, an outer loop and an inner loop may be set based on the entire training data set that may be used for the prediction model, evaluation of the model may be performed in the outer loop, and tuning of the hyper parameter may be performed in the inner loop. For example, after dividing the entire training data set into r groups in the outer loop, the values of the hyper parameters used in the machine learning model are input and cross-verified to attempt all possible hyper parameter combinations in the inner loop, and training and evaluation for each group may be performed in the outer loop to determine an optimal parameter combination.

Meanwhile, various known evaluation methods may be used to measure the evaluation of the prediction performance of the model. For example, in the case of the regression model, an MSE evaluation method (e.g., neg_mean_squared_error) may be used. In addition, in the case of the classification model, a receiver operating characteristic (ROC), an area under the ROC curve (AUC), ROC-AUC, ACU_SD, Specificity, F1, a negative predictive value (NPV)-based evaluation method, and the like may be used.

According to an embodiment, by using the above-described Nested CVmethod, there is an advantage in that problems such as overfitting for test sets and dependence due to data division, which may occur in a method of verifying the learning of a model by dividing the training and test sets only once, may be solved. In addition, the cross-validation method according to an embodiment has an advantage in that the prediction accuracy of the model increases as the learning progresses for all training data sets.

Table 2 below shows performance evaluation scores of the prediction model 910 supervised based on the regression model according to an embodiment. In Table 2, in the evaluation of the prediction model, 5-fold was applied in the outer and inner loops based on K-old, respectively, and the average values of the evaluation indexes in five folds for the scores of the test set of the outer fold were described. In addition, in Table 2, AUC for each case is described according to an exemplary performance evaluation method, but various evaluation methods known in the art may be used as described above.

TABLE 2

| | the first | the second | the third | the fourth |
|---|---|---|---|---|
| | prediction | prediction | prediction | prediction |
| | model | model | model | model |
| Case 1 | 0.6472 | 0.6798 | 0.6820 | 0.6792 |
| Case 2 | 0.8378 | 0.8877 | 0.8477 | 0.8671 |
| Case 3 | 0.8330 | 0.8791 | 0.8649 | 0.8351 |
| Case 4 | 0.8854 | 0.9415 | 0.9435 | 0.9223 |
| Case 5 | 0.8676 | 0.9433 | 0.9460 | 0.9047 |
| Case 6 | 0.8780 | 0.9458 | 0.9448 | 0.9453 |
| Case 7 | 0.8753 | 0.9523 | 0.9429 | 0.9347 |
| Case 8 | 0.7017 | 0.8467 | 0.7157 | 0.8247 |
| Case 9 | 0.8824 | 0.9478 | 0.9390 | 0.9132 |
| Case 10 | 0.8941 | 0.9450 | 0.9318 | 0.9399 |
| Case 11 | 0.8763 | 0.9418 | 0.9450 | 0.9308 |

Table 2 shows various cases in which the learning was performed by using various type of the training input data, for example, a first case (Case 1) using the Tm of the primer, a second case (Case 2) using the GC content, a third case (Case 3) using the Tm of the primer and the GC content, a fourth case (Case 4) using the amount of change in the Gibbs free energy of the secondary structure in the nucleic acid sequence, a fifth case (Case 5) using the Tm of the primer and the amount of change in the Gibbs free energy, a sixth case (Case 6) using the GC content and the amount of change in the Gibbs free energy, a seventh case (Case 7) using the Tm of the primer, the GC content, and the amount of change in the Gibbs free energy, an eighth case (Case 8) using the G-quadruplex score, a ninth case (Case 9) using the amount of change in the Gibbs free energy and the G-quadruplex score, a tenth case (Case 10) using the GC content, the amount of change in the Gibbs free energy, and the G-quadruplex score, and an eleventh case (Case 11) using the Tm of the primer, the GC content, the amount of change in the Gibbs free energy, and the G-quadruplex score. In addition, with respect to each of the above cases, evaluation scores in a case in which learning is performed by using each model are shown, for example, a first prediction model based on ridge linear regression analysis, a second prediction model based on random forest regression, a third prediction model based on logistic classification, and a fourth prediction model based on random forest classification.

As can be seen from Table 2, it can be seen that the cases using the amount of change in the Gibbs free energy as the training input data has a higher evaluation score than the other cases. In addition, it can be seen that the accuracy is further improved when the above-described amount of change in the Gibbs free energy is used as an independent variable along with other parameters as the training input data. In addition, high evaluation scores were obtained in cases 9 to 11 in which the G-quadruplex score were used together as the training input data. Although Table 1 shows the AUC as an example, it was confirmed that not only the AUC but also the evaluation case using other evaluation methods showed the average high evaluation score with respect to the amount of change in the Gibbs free energy compared with other variables.

As described above, by using the above-described amount of change in the Gibbs free energy, it is possible to more accurately predict for cases in which the amplification reaction is inhibited by the formation of an arbitrary nth-order structure. In addition, by using the above-described G-quadruplex score, it is possible to accurately predict for cases in which the amplification reaction is inhibited by the formation of the G-quadruplex.

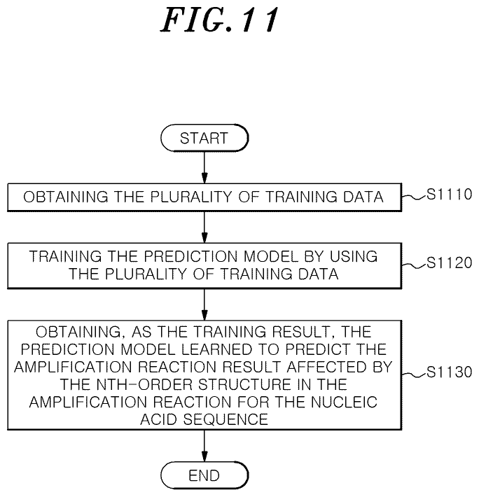

FIG. 11 illustrates an example flowchart for obtaining the prediction model by the computer device 1000 according to an embodiment. In one embodiment, the steps of FIG. 11 may be implemented by one entity, such as in the manner performed by the server. In another embodiment, the steps of FIG. 11 may be implemented by a plurality of entities, such as in the manner some of the steps of FIG. 11 performed by the user terminal and the others performed by the server.

Referring to FIG. 11, in step S1110, the computer device 1000 may obtain the plurality of training data. As described above, each training data may include the first analysis data for the nth-order structure in the nucleic acid sequence and the amplification reaction result for the corresponding nucleic acid sequence, and may further include at least one selected from the group consisting of (a) at least one of the nucleic acid sequence, the amplicon sequence obtained from the nucleic acid sequence, and the oligonucleotide sequence bound to the nucleic acid sequence; (b) the Tm of at least one of the nucleic acid sequence, the amplicon sequence, and the oligonucleotide sequence; (c) the length of at least one of the nucleic acid sequence, the amplicon sequence, and the oligonucleotide sequence; (d) the type of the nucleic acid sequence; and (e) the GC content of at least one of the nucleic acid sequence, the amplicon sequence, and the oligonucleotide sequence.

In step S1120, the computer device 1000 may train the prediction model by using the plurality of training data. In an embodiment, the prediction model may include at least one of the machine learning-based regression analysis model and the classification model, and may be at least one selected from the group consisting of, for example, the Ridge linear regression model, the random forest regression model, the logistic regression model, and the random forest classification model.

In step S1130, the computer device 1000 may obtain, as the training result, the prediction model learned to predict the amplification reaction result affected by the nth-order structure in the amplification reaction for the nucleic acid sequence. As described above, the amplification reaction result may include at least one selected from the group consisting of (a) the amplification inhibition level representing a level at which the amplification reaction is inhibited by the nth-order structure, and (b) the amplification inhibition or the amplification non-inhibition representing whether the amplification inhibition level satisfies a predetermined criterion.

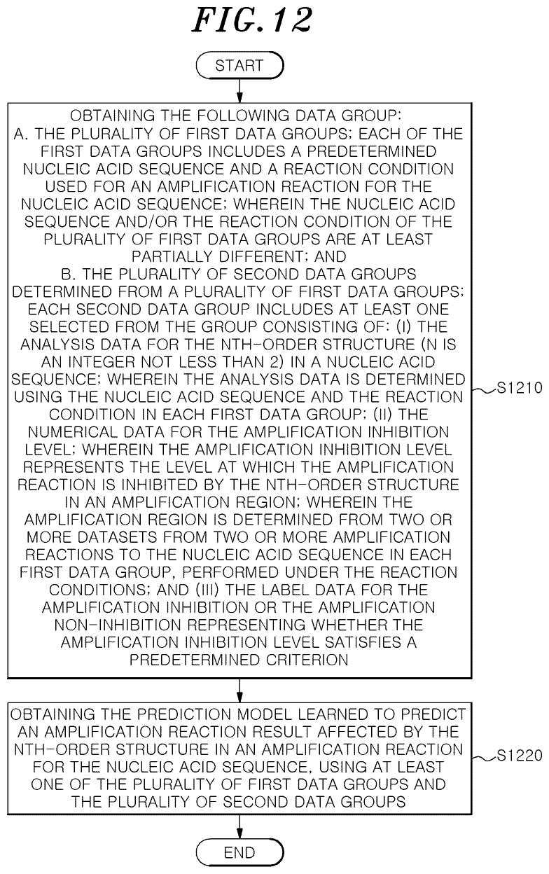

FIG. 12 illustrates an example flowchart for obtaining the prediction model by the computer device 1000 according to another embodiment. Similarly, the steps of FIG. 12 may be implemented by one entity, such as in the method performed by the server, or the steps may be implemented by the plurality of entities, such as in the method some performed by the user terminal and others by the server.

Referring to FIG. 12, in step S1210, the computer device 1000 may obtain the following data group:

a. the plurality of first data groups; each of the first data groups includes a predetermined nucleic acid sequence and a reaction condition used for an amplification reaction for the nucleic acid sequence; wherein the nucleic acid sequence and/or the reaction condition of the plurality of first data groups are at least partially different; and b. the plurality of second data groups determined from a plurality of first data groups; each second data group includes at least one selected from the group consisting of: (i) the analysis data for the nth-order structure (n is an integer not less than 2) in a nucleic acid sequence; wherein the analysis data is determined using the nucleic acid sequence and the reaction condition in each first data group; (ii) the numerical data for the amplification inhibition level; wherein the amplification inhibition level represents the level at which the amplification reaction is inhibited by the nth-order structure in an amplification region; wherein the amplification region is determined from two or more datasets from two or more amplification reactions to the nucleic acid sequence in each first data group, performed under the reaction conditions; and (iii) the label data for the amplification inhibition or the amplification non-inhibition representing whether the amplification inhibition level satisfies a predetermined criterion.

In step S1220, the computer device 1000 may obtain the prediction model learned to predict an amplification reaction result affected by the nth-order structure in an amplification reaction for the nucleic acid sequence, using at least one of the plurality of first data groups and the plurality of second data groups.

As described above, the prediction model learned through the learning process may be obtained. The computer device 1000 may store and manage the prediction model, and may provide the prediction model. For example, the computer device 1000 may be implemented to store and manage the prediction model trained by the server, and may be implemented to provide the prediction model to the user terminal when the user terminal requests the prediction model.

### Obtaining Process of Input Data to be Provided to the Prediction Model

The prediction unit 120 according to an embodiment may obtain an input data to be provided to the prediction model. The input data according to an embodiment may include a second analysis data for an nth-order structure in a target nucleic acid sequence. The obtaining process of the input data may be performed in a manner similar to the obtaining process of the training input data and the preprocessing process in the corresponding process described above, and a redundant description thereof will be omitted.

The input data according to an embodiment may include the second analysis data for an nth-order structure present in a predetermined target nucleic acid sequence. Similarly, in an embodiment, the second analytical data for the nth-order structure may include a thermodynamic data for a formation of a specific nth-order structure in the target nucleic acid sequence, for example, at least one selected from the group consisting of the thermodynamic data of the first block unit 20a to the thermodynamic data of the third block unit 20c for the target nucleic acid sequence. In another embodiment, the second analytical data may include at least one of the value of n, the type of the nth-order structure, the characteristic of the nth-order structure, and information about whether the nth-order structure is formed. In another embodiment, the second analytical data may include the analysis data for the structural characteristics of a specific nth-order structure appearing at bases in the nucleic acid sequence, for example, the G-quadruplex score for the structural characteristics of the G-quadruplex appearing at the bases. In addition, according to an embodiment, the input data may further include at least one of at least one of the target nucleic acid sequence, the amplicon sequence obtained from the target nucleic acid sequence, and the oligonucleotide sequence bound to the target sequence, the Tm, the length, the type, and the GC content.

In an embodiment, the prediction unit 120 may obtain at least one of a predetermined target nucleic acid sequence and a reaction condition used for an amplification reaction for the corresponding target nucleic acid sequence, and may obtain the above-described input data from the obtained information. For example, the prediction unit 120 may load the information about the target nucleic acid sequence and the corresponding reaction condition from the memory 100 or the storage device, receive a user input for the information, or receive the information from another device through the communication unit 200. Also, the prediction unit 120 may determine the first block unit 20a to the third block unit 20c in the target nucleic acid sequence based on the target nucleic acid sequence and the reaction condition, and may calculate the amount of change in the first Gibbs free energy (ΔG₁) to the amount of change in the third Gibbs free energy (ΔG₃) and the length of the first block unit 20a to the third block unit 20c and GC content based on the first block unit 20a to the third block unit 20c. In addition, the prediction unit 120 may calculate the G-quadruplex score by applying the target nucleic acid sequence and the amplicon sequence determined from the target nucleic acid sequence to the previously stored G-quadruplex score calculation algorithm. In addition, the prediction unit 120 may apply the target nucleic acid sequence and the corresponding oligonucleotide sequence (e.g., forward primer sequence, reverse primer sequence) to the pre-stored Tm prediction algorithm to calculate the Tm of the corresponding oligonucleotide sequence.

In another embodiment, the prediction unit 120 may obtain the second analysis data for the nth-order structure present in the predetermined target nucleic acid sequence, which is previously prepared, and may obtain the input data comprising the second analysis data. For example, the prediction unit 120 may load the values of each feature included in the input data from the memory 100 or the storage device, receive a user input for the corresponding value, or receive the values from another device through the communication unit 200.

The obtaining process of the input data according to an embodiment may be implemented by one entity, such as in the method performed by the user terminal. According to another embodiment, the obtaining processes of the input data may be implemented by the plurality of entities, such as in the manner some performed by the user terminal and others performed by the server.

### Obtaining Process of a Prediction Result of a Nucleic Acid Amplification Reaction Affected by an Nth-Order Structure Using the Prediction Model

The prediction unit 120 according to an embodiment may obtain a prediction result of an amplification reaction for the target nucleic acid sequence by using the prediction model. Specifically, the prediction unit 120 may provide the input data to the prediction model to obtain, from the prediction model, a prediction result of the amplification reaction affected by the nth-order structure in the amplification reaction for the target nucleic acid sequence. The prediction unit 120 may input the input data to the prediction model, and obtain the prediction result of the amplification reaction output from the prediction model in response to the input data. As described above, the prediction result represents (i) a level (or an extent) at which the amplification reaction for the target nucleic acid sequence is affected by a formation of the nth-order structure in the target nucleic acid sequence and/or (ii) whether the amplification reaction for the target nucleic acid sequence is affected or not by the formation of the nth-order structure in the target nucleic acid sequence. The obtaining process of the prediction result may be performed in a similar manner to the process of obtaining the output result according to the input by the prediction model 910 in the learning process of the prediction model 910 described above, and similarly, redundant contents will be omitted.

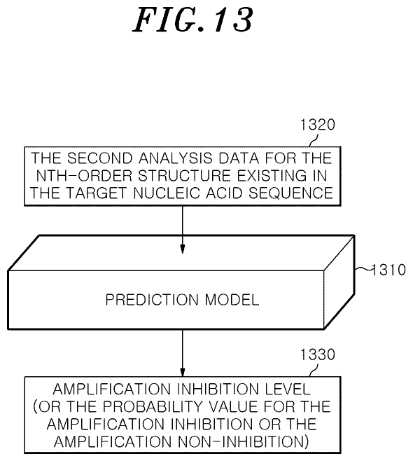

FIG. 13 exemplarily illustrates a conceptual diagram a process in which a prediction model 1310 outputs the prediction result of the nucleic acid amplification reaction affected by the nth-order structure, according to an embodiment. Here, the prediction model 1310 may correspond to the prediction model 910 on which the learning is performed.

Referring to FIG. 13, when the input data including the second analysis data 1320 for the nth-order structure existing in the target nucleic acid sequence is input, the prediction model 1310 may output an amplification inhibition level (or a probability value for the amplification inhibition or the amplification non-inhibition) 1330 by the nth-order structure in the target nucleic acid sequence as a prediction result. As described above, the prediction model 1310 may be learned to output the amplification inhibition level corresponding to the dependent variable when the second analysis data corresponding to the independent variable is input. Accordingly, the prediction model 1310 may output the corresponding amplification inhibition level or the amplification inhibition (or the amplification non-inhibition) from the input data based on the relationship between the dependent variable and the independent variable in the prediction function implemented through the learning.

In an embodiment, the prediction result may include at least one selected from the group consisting of (a) the amplification inhibition level representing a level at which the amplification reaction for the target nucleic acid sequence is inhibited by the nth-order structure, (b) the amplification inhibition or the amplification non-inhibition representing whether the amplification inhibition level satisfies the predetermined criterion, and (c) a probability value for the amplification inhibition or the amplification non-inhibition. For example, the amplification inhibition level by the formation of the nth-order structure in the target nucleic acid sequence corresponding to any one of the first amplification inhibition level 810a to the third amplification inhibition level 810c may be output as a numerical value from the prediction model.

In an embodiment, the prediction result may include the corresponding amplification inhibition level and the amplification inhibition (or the amplification non-inhibition). For example, the prediction unit 120 may output the calculated amplification inhibition level, the predetermined criterion (e.g., a reference value), and whether the corresponding amplification inhibition level satisfies the predetermined criterion as the prediction result.

In another embodiment, the prediction result may include one or more classes representing a section to which the corresponding amplification inhibition level belongs. For example, a first class indicating that the amplification will be inhibited and a second class indicating that the amplification will not be inhibited are preset, and the prediction unit 120 may output the first class or the second class as the prediction result according to whether the calculated amplification inhibition level is equal to or greater than the reference value. For another example, a plurality of sections corresponding to a plurality of amplification inhibition intensities (e.g., strong inhibition, medium inhibition, weak inhibition, and no inhibition) and a plurality of classes corresponding to the plurality of section are preset, and the prediction unit 120 may output information about each section (e.g., a section name, a range of the amplification inhibition level set for each section, etc.) and a probability value for each class that the amplification inhibition level belongs to each section as the prediction result.

In another embodiment, the prediction result may include a probability value for each of the plurality of classes. For example, the prediction unit 120 may output the probability value for each of the first class and the second class as a prediction result, or may output any one class and probability value having the greatest probability value among them or satisfying a preset criterion (e.g., a threshold for the probability value).

In another embodiment, the prediction result may include information about the nth-order structure related to the amplification inhibition. For an example, when amplification inhibition is predicted, the prediction unit 120 may provide a description or an image about the type of the nth-order structure (e.g., hairpin) expected to cause the corresponding amplification inhibition among the plurality of types of n-order structures, based on (i) the thermodynamic data for the formation of each of the plurality of types of nth-order structures in the input data, or (ii) a comparison result between the values of the independent variables in the input data and the pre-stored reference ranges.

The prediction unit 120 may provide the above-described prediction result in various ways. For example, the prediction unit 120 may output a result screen including information about the target nucleic acid sequence and the nth-order structure and the prediction result. In addition, the prediction unit 120 may output the result screen including the probability values for each class, and may output the result screen so that the probability values in the result screen are arranged in an order from the highest to the lowest probability value or in an order from the lowest to the highest probability value. In addition, the prediction unit 120 may output the result screen in which a probability value of a class satisfying a preset criterion is highlighted.

The result screen may be displayed in the form of a table, a graph, or an image according to the implementation aspect, and the type, scale, or the like of the table or the graph may be different.

The prediction unit 120 may provide a contribution level for the above prediction result. As described above, a plurality of feature (e.g., ΔG_{1A}, ΔG_{1B}, ΔG₂, ΔG₃, L_{1A}, L_{1B}, L₂, and L₃) may be included in the input data, and the prediction unit 120 may calculate the contribution level representing a level to which each of the plurality of feature contributes to the prediction result by applying a pre-stored contribution calculation method. For example, as the contribution calculation method, a method of checking coefficients of independent variables defining a prediction function implemented in a model, a method of checking a classification weight of a tree included in the model, a method of checking a feature, a weight, a main object position, and the like, of input data dependent on the model (e.g., LRP, and the like), a method of analyzing a cause while viewing an output obtained by adjusting an input without being dependent on the model (e.g., LIME), a method of extracting an explainable feature from the model (e.g., SmoothGrad, and the like), and the like may be used.

In an embodiment, when the prediction result is obtained, the prediction unit 120 may calculate and provide the contribution level of each feature used for the prediction result. In another embodiment, when the prediction model 1310 is obtained, the prediction unit 120 may calculate and provide the contribution level of each feature to the prediction model 1310.

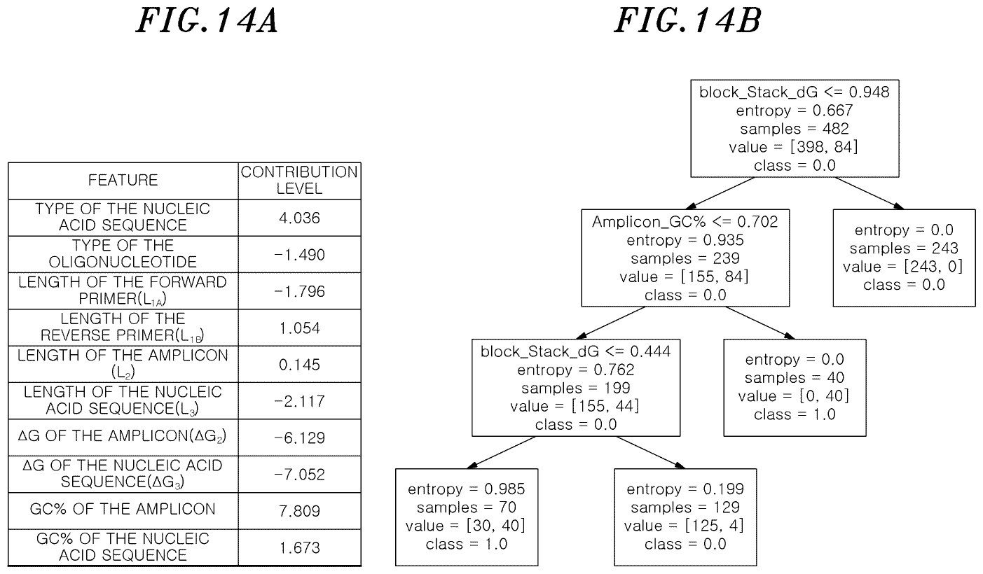

FIGS. 14A and 14B show a diagram illustrating an operation of providing a contribution level by the computer device 1000 according to an embodiment.

Referring to FIGS. 14A and 14B, the prediction unit 120 may output a comparison screen including contribution levels of a plurality of features. The contribution levels may be output in various ways, for example, in the form of various chart graphs.

FIG. 14A illustrates the contribution level of each feature in the prediction model 1310 implemented as the logistic regression-based classification model. In this way, the contribution of each feature may be processed in a comparable form and displayed on one screen. In FIG. 14A, the contribution level was calculated to be greater in the negative direction as the contribution level increased. It can be seen that the contribution levels of the amount of change in the Gibbs free energy and the GC content of amplicon are relatively greater than the contribution levels of other features.

FIG. 14B illustrates the contribution level of each feature in the prediction model 1310 implemented as the decision tree classification model. As described above, the structure of the plurality of nodes (e.g., a decision node, an opportunity node, and an end node) included in the decision tree, a variable and a classification condition that are classification criteria in each node, an entropy obtained in a classification operation, a classification frequency according to a class, and the like may be displayed on one screen as the contribution level of the corresponding feature (ex. the variable that is the classification criterion).

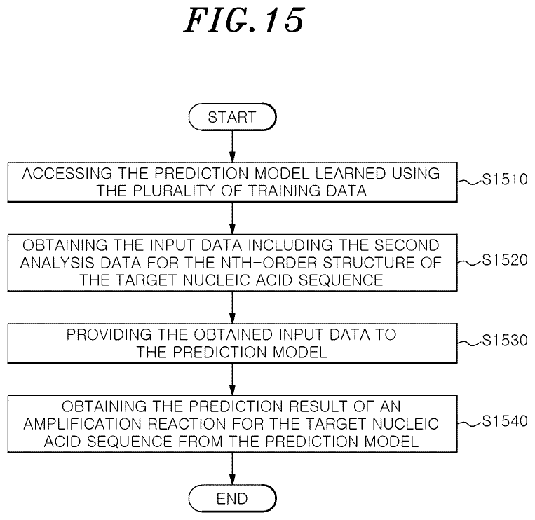

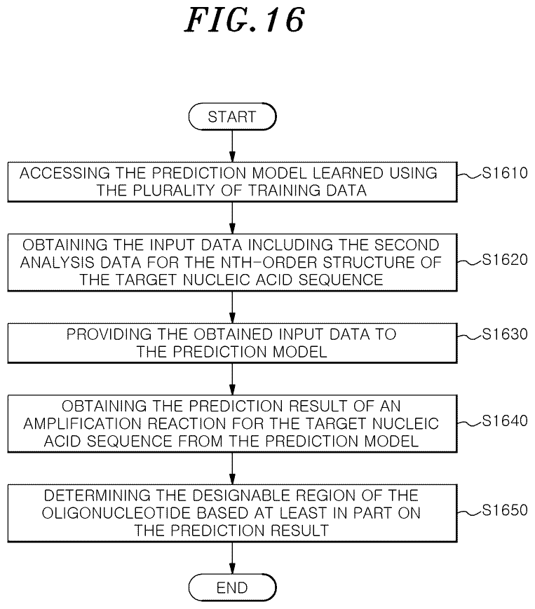

FIG. 15 illustrates an exemplary flowchart for obtaining the prediction result of the nucleic acid amplification reaction affected by the nth-order structure, by the computer device 1000 according to an embodiment. In an embodiment, the steps of FIG. 15 may be implemented by one entity, such as in a manner performed by a user terminal. In another embodiment, the steps of FIG. 15 may be implemented by the plurality of entities, such as a method some performed by the user terminal and the others performed by the server.

Referring to FIG. 15, in step S1510, the computer device 1000 may access the prediction model 1310 learned using the plurality of training data.

In an embodiment, the computer device 100 may be implemented to access the prediction model 1310 in a manner that the user terminal receives and executes the prediction model 1310 learned by the server from the server. In another embodiment, the computer device 100 may be implemented to access the prediction model 1310 in such a manner that the prediction model 1310 trained by the server is stored in the database and the user terminal accesses the database to receive and execute the prediction model 1310. In another embodiment, the computer device 100 may be implemented to access the prediction model 1310 in a manner that the user terminal loads and executes the prediction model 1310 previously stored in the memory 110 or another storage medium. In another embodiment, the computer device 100 may be implemented to access the prediction model 1310 in such a manner that the user terminal transmits a request for execution of the prediction model 1310 learned by the server and data (e.g., input data, etc.) required for executing the prediction model 1310 to the server and receives the execution result of the prediction model 1310 from the server. However, the access to the prediction model 1310 in the present disclosure is not limited thereto, and may be variously modified and implemented. In addition, as described above, the prediction model 1310 according to an embodiment may include at least one selected from the group consisting of the machine learning-based Ridge linear regression model, the random forest regression model, the logistic regression-based classification model, and the random forest classification model.

In step S1520, the computer device 1000 may obtain the input data comprising the second analysis data for the nth-order structure of the target nucleic acid sequence. As described above, the input data includes the second analysis data for the nth-order structure in the target nucleic acid sequence, and may further includes at least one selected from the group consisting of (a) at least one of the target nucleic acid sequence, the amplicon sequence obtained from the target nucleic acid sequence, and the oligonucleotide sequence bound to the target nucleic acid sequence; (b) the Tm of at least one of the target nucleic acid sequence, the amplicon sequence, and the oligonucleotide sequence; (c) the length of at least one of the target nucleic acid sequence, the amplicon sequence, and the oligonucleotide sequence; (d) the type of the target nucleic acid sequence; and (e) the GC content of at least one of the target nucleic acid sequence, the amplicon sequence, and the oligonucleotide sequence.

In step S1530, the computer device 1000 may provide the obtained input data to the prediction model 1310. The computer device 1000 may input the input data obtained in step S1520 to the prediction model 1310, and the prediction model 1310 may perform the process of predicting the result of the nucleic acid amplification reaction affected by the nth-order structure by using the input data.

In step S1540, the computer device 1000 may obtain the prediction result of an amplification reaction for the target nucleic acid sequence from the prediction model 1310. As described above, the above-described prediction result is a prediction result of the amplification reaction affected by the nth-order structure in the amplification reaction, and may include at least one selected from the group consisting of (a) the amplification inhibition level representing a level at which the amplification reaction is inhibited by the nth-order structure, (b) the amplification inhibition or the amplification non-inhibition representing whether the amplification inhibition level satisfies the predetermined criterion, and (c) the probability value for the amplification inhibition or the amplification non-inhibition.

As described above, the method for obtaining the prediction result of the nucleic acid amplification reaction affected by the nth-order structure may be performed by using the prediction model described in the present specification. These technical features may be used independently without being combined with the method for obtaining the prediction model, according to an embodiment.

### Determining Process of a Designable Region of an Oligonucleotide

In the above, the embodiments for obtaining the prediction result of the nucleic acid amplification reaction affected by the nth-order structure have been mainly described. However, the present disclosure is not limited thereto, and based on the technical idea described above, the designable region determination unit 130 may perform a determining process of a designable region of an oligonucleotide.

When a conserved position and a non-conserved position with respect to a target nucleic acid sequence are determined in a manner of aligning a plurality of known nucleic acid sequences through a published database such as NCBI or GISAID, a position at which an amplification reaction is likely to be inhibited by an nth-order structure may be determined as the conserved position. In this case, an oligonucleotide for detecting the target nucleic acid sequence may be designed based on the region determined as the conserved region. If the amplification reaction is inhibited by the nth-order structure at the corresponding conserved position after the oligonucleotide is designed, the detection performance for the target nucleic acid sequence of the designed oligonucleotide may be degraded.

According to an embodiment of the present disclosure, it is possible to predict the amplification inhibition level or the amplification inhibition (or the amplification non-inhibition), in which the amplification reaction for the target nucleic acid sequence will be inhibited by the nth-order structure, by using the prediction model trained based on the data for the nth-order structure present in the nucleic acid sequence. Accordingly, the prediction for the amplification inhibition level or the amplification inhibition (or the amplification non-inhibition) may be sufficiently considered when the oligonucleotide is designed. The designable region determination unit 130 may design the oligonucleotide by considering an unstable region of a sequence in which such the nth-order structure may be formed or the amplification reaction may be inhibited thereby.

In an embodiment of the present disclosure, the designable region determination unit 130 may determine the designable region of the oligonucleotide at least in part on the prediction result. Specifically, the designable region determination unit 130 may determine the conserved region of the oligonucleotide in the reference nucleic acid sequence based on the output of the prediction model. Since the possibility of the amplification inhibition by the formation of the nth-order structure of a specific nucleic acid sequence in the reference nucleic acid sequence may be considered according to the above prediction result, the oligonucleotide designed based on the conserved region determined in this way may have more robust performance. As an example, when an oligonucleotide designed according to an embodiment of the present disclosure is used, the possibility of a false positive result may be reduced.

According to an embodiment of the present disclosure, since a position at which the amplification inhibition level by the nth-order structure is predicted to be relatively high or the amplification is predicted to be inhibited, among conserved positions, may be treated in accordance with a non-conserved position, an oligonucleotide specific to a specific organism may be designed in consideration of a position at which the amplification inhibition level by the nth-order structure is relatively low or the amplification is not inhibited.

Here, the reference nucleic acid sequence may include a unique genome sequence corresponding to an organism. In an embodiment, the reference nucleic acid sequence may mean a sequence that is a representative of a specific species or individual. For example, the reference nucleic acid sequence may refer to a sequence that appears first in the specific species or the individual. In another example, the reference nucleic acid sequence may be determined based on sequence accuracy and assembly quality for the species or the individual. For example, the reference nucleic acid sequence may be GRCh38 for human, GRCm39 for mouse, and canFam5 for dog. The description of the reference nucleic acid sequences corresponding to these individuals has been used for illustrative purposes and the reference nucleic acid sequence may vary over time in a specific species.

In an embodiment, one or more target nucleic acid sequences may be determined based on the reference nucleic acid sequence corresponding to the target analyte, and the designable region of the oligonucleotide may be determined based on the above-described prediction result obtained for each target nucleic acid sequence. For example, in order to effectively detect the target analyte, one or more target nucleic acid sequences each composed of some of the bases included in the reference nucleic acid sequence corresponding to the target analyte may be determined. In addition, the amplification inhibition level or the amplification inhibition by the nth-order structure may be predicted for each target nucleic acid sequence by using the prediction model, and based on these prediction results, an oligonucleotide specifically hybridizing to the target nucleic acid sequence in which the amplification reaction is predicted not to be inhibited by the nth-order structure may be designed.

In an embodiment, the designable region determination unit 130 may determine the position of the target nucleic acid sequence at which the amplification inhibition level or the probability value of the amplification inhibition by the nth-order structure is not less than a predetermined reference value as the non-conserved position. The designable region determination unit 130 may determine the designable region of the oligonucleotide such that the number of non-conserved positions included in the designable region is minimized. As an example, the designable region may mean a region used when designing an oligonucleotide among regions in which a plurality of bases included in the reference nucleic acid sequence are consecutive. The designable region determination unit 130 may determine the designable region of the oligonucleotide based on the amplification inhibition level or the probability value of the amplification inhibition of each of the plurality of target nucleic acid sequences such that the number of target nucleic acid sequences having the amplification inhibition level of 2 or more or the probability value of the amplification inhibition of 50% or more is less than a minimum or specific number.

In an embodiment, the designable region determination unit 130 may obtain one or more candidate regions composed of some of the plurality of bases included in the reference nucleic acid sequence, as candidate regions that are targets of the designable region. In addition, the designable region determination unit 130 may determine a region to be excluded from the one or more candidate regions based on the prediction result obtained from the prediction model.

As a specific example, the designable region determination unit 130 may exclude bases corresponding to target nucleic acid sequences for which the amplification inhibition level or the probability value of the amplification inhibition is not less than the reference value from bases included in the reference nucleic acid sequence, and then identify at least one region which is consecutively arranged among the bases that are not excluded. In addition, the designable region determination unit 130 may determine the identified at least one region as the designable region of the oligonucleotide. For example, the designable region determination unit 130 may remove bases corresponding to the target nucleic acid sequence having the amplification inhibition level not less than 2 or the probability value of the amplification inhibition not less than 80% from the reference nucleic acid sequence. The designable region determination unit 130 may select a region that may correspond to the length of the oligonucleotide (e.g., 15 to 30 bases) within the reference nucleic acid sequence from which these bases have been removed. In such examples, the above-described reference value may be variably determined based at least in part on length information of the oligonucleotide, performance information of the oligonucleotide, the value of n and/or type information of the nth-order structure.

In an embodiment, the designable region determination unit 130 may a generate annotation with respect to the probability value of the amplification inhibition by the nth-order structure for each section of bases in the reference nucleic acid sequence representing the representative sequence of the specific species, and determine the nucleic acid sequence of the oligonucleotide by using the generated annotation information to avoid one or more oligo binding sites in which there is a high probability of the amplification inhibition.

FIG. 16 illustrates an exemplary flowchart for determining the designable region of the oligonucleotide by the computer device 1000 according to an embodiment. FIG. 16 may be understood with reference to the embodiments described above.

Referring to FIG. 16, steps S1610 to S1640 may be performed in the same manner as steps S1510 to S1540 described above. In addition, in step S1650, the computer device 1000 may determine the designable region of the oligonucleotide based at least in part on the prediction result.

These technical features may be used independently without being combined with the method of obtaining the prediction model, according to an embodiment.

According to one aspect of the present invention, the computer device 1000 is provided. The computer device 1000 includes the memory 100 configured to store at least one instruction; and the processor 300, wherein the at least one instruction is executed by the processor 300 to: access a prediction model learned using a plurality of training data; each training data includes a first analysis data for an nth-order structure in a nucleic acid sequence and an amplification reaction result for the nucleic acid sequence; wherein n is an integer not less than 2; obtain an input data comprising a second analysis data for an nth-order structure in a target nucleic acid sequence; provide the input data to the prediction model; and obtain a prediction result of an amplification reaction for the target nucleic acid sequence from the prediction model.

Since each component described in the above-described aspect of the present invention overlaps with the method for obtaining the prediction result of the amplification reaction described with reference to FIGS. 1 to 16, the description thereof is omitted.

According to one aspect of the present invention, a computer-readable recording medium storing a computer program is provided. The computer program includes an instruction that, when executed by one or more processors 300, enables the one or more processors 300 to perform a method for obtaining a prediction result of a nucleic acid amplification reaction affected by an nth-order structure, the method comprising: accessing a prediction model learned using a plurality of training data; each training data includes a first analysis data for an nth-order structure in a nucleic acid sequence and an amplification reaction result for the nucleic acid sequence; wherein n is an integer not less than 2; obtaining an input data comprising a second analysis data for an nth-order structure in a target nucleic acid sequence; providing the input data to the prediction model; and obtaining a prediction result of an amplification reaction for the target nucleic acid sequence from the prediction model.

Since each component described in the above-described aspect of the present invention overlaps with the method for obtaining the prediction result of the amplification reaction described with reference to FIGS. 1 to 16, the description thereof is omitted.

According to one aspect of the present invention, a computer program stored in a computer-readable recording medium is provided. The computer program is programmed to perform each step in a method for obtaining a prediction result of a nucleic acid amplification reaction affected by an nth-order structure, the method comprising: accessing a prediction model learned using a plurality of training data; each training data includes a first analysis data for an nth-order structure in a nucleic acid sequence and an amplification reaction result for the nucleic acid sequence; wherein n is an integer not less than 2; obtaining an input data comprising a second analysis data for an nth-order structure in a target nucleic acid sequence; providing the input data to the prediction model; and obtaining a prediction result of an amplification reaction for the target nucleic acid sequence from the prediction model.

Since each component described in the above-described aspect of the present invention overlaps with the method for obtaining the prediction result of the amplification reaction described with reference to FIGS. 1 to 16, the description thereof is omitted.

According to one aspect of the present invention, the computer device 1000 is provided. The computer device 1000 includes the memory 100 configured to store at least one instruction; and the processor 300, wherein the at least one instruction is executed by the processor 300 to: obtain the following data group: a. a plurality of first data groups; each first data group comprising a predetermined nucleic acid sequence and a reaction condition used for an amplification reaction for the nucleic acid sequence; wherein the nucleic acid sequence and/or the reaction condition of the plurality of first data groups are at least partially different; and b. a plurality of second data groups determined from the plurality of first data groups; each second data group comprising at least one selected from the group consisting of: (i) an analysis data for an nth-order structure in the nucleic acid sequence; wherein n is an integer not less than 2; wherein the analysis data is determined using the nucleic acid sequence and the reaction condition in each first data group; (ii) a numerical data for an amplification inhibition level; wherein the amplification inhibition level represents a level at which the amplification reaction is inhibited by the nth-order structure in an amplification region; wherein the amplification region is determined from two or more datasets obtained from two or more amplification reactions to the nucleic acid sequence in each first data group, performed under the reaction condition, and (iii) a label data for an amplification inhibition or an amplification non-inhibition representing whether the amplification inhibition level satisfies a predetermined criterion; and obtain a prediction model learned to predict an amplification reaction result for the nucleic acid sequence, using at least one of the plurality of first data groups and the plurality of second data groups.

Since each component described in the above-described aspect of the present invention overlaps with the method for obtaining the prediction model described with reference to FIGS. 1 to 16, the description thereof is omitted.

According to one aspect of the present invention, a computer-readable recording medium storing a computer program is provided. The computer program includes an instruction that, when executed by one or more processors 300, enables the one or more processors 300 to perform a method for obtaining a prediction model providing a prediction result of a nucleic acid amplification reaction affected by an nth-order structure, the method comprising: obtaining the data group comprising the plurality of first data groups and the plurality of second data groups; and obtaining a prediction model learned to predict an amplification reaction result for the nucleic acid sequence, using at least one of the plurality of first data groups and the plurality of second data groups.

Since each component described in the above-described aspect of the present invention overlaps with the method for obtaining the prediction model described with reference to FIGS. 1 to 16, the description thereof is omitted.

According to one aspect of the present invention, a computer program stored in a computer-readable recording medium is provided. The computer program is programmed to perform each step in a method for obtaining a prediction model providing a prediction result of a nucleic acid amplification reaction affected by an nth-order structure, the method comprising: obtaining the data group comprising the plurality of first data groups and the plurality of second data groups; and obtaining a prediction model learned to predict an amplification reaction result for the nucleic acid sequence, using at least one of the plurality of first data groups and the plurality of second data groups.

Since each component described in the above-described aspect of the present invention overlaps with the method for obtaining the prediction model described with reference to FIGS. 1 to 16, the description thereof is omitted.

On the other hand, those of ordinary skill in the art will understand that the various illustrative logical blocks, modules, processors, means, circuits, and algorithm steps described in connection with the embodiments disclosed herein may be implemented by electronic hardware, various forms of program or design code (referred to herein, for convenience, as software), or a combination of both. To clearly illustrate this interchangeability of hardware and software, various illustrative components, blocks, modules, circuits, and steps have been described generally above in relation to their functionality. Whether this functionality is implemented as hardware or software depends on design constraints imposed on the particular application and the overall system. A person having ordinary skill in the art of the present disclosure may implement the described functions in various ways for each specific application, but such implementation decisions should not be interpreted as being out of the scope of the present disclosure.

Various embodiments presented herein may be implemented in a method, device, or article of manufacture using standard programming and/or engineering techniques. The term article of manufacture includes a computer program, carrier, or media accessible from any computer-readable storage device. For example, the computer-readable storage medium includes a magnetic storage device (e.g., a hard disk, a floppy disk, a magnetic strip, etc.), an optical disk (e.g., a CD, a DVD, etc.), a smart card, and a flash memory device (e.g., a EEPROM, a card, a stick, a key drive, etc.), but is not limited thereto. In addition, the various storage media presented herein include one or more devices and/or other machine-readable media for storing information.

It should be understood that the specific order or hierarchy of steps in the presented processes is an example of exemplary approaches. Based on design priorities, it should be understood that the specific order or hierarchy of steps in the processes may be rearranged within the scope of the present disclosure. The appended method claims provide the elements of the various steps in a sample order, but are not meant to be limited to the particular order or hierarchy presented.

What is claimed is:

1. A method for obtaining a prediction result of a nucleic acid amplification reaction affected by an nth-order structure, performed by a computing device, the method comprising:
accessing a prediction model learned using a plurality of training data; each training data comprises a first analysis data for an nth-order structure in a nucleic acid sequence and an amplification reaction result for the nucleic acid sequence; wherein n is an integer not less than 2;
obtaining an input data comprising a second analysis data for an nth-order structure in a target nucleic acid sequence;
providing the input data to the prediction model; and
obtaining a prediction result of an amplification reaction for the target nucleic acid sequence from the prediction model.

2. The method of claim 1, wherein n is 2, and wherein the nth-order structure comprises at least one selected from the group consisting of:
a hairpin loop, an internal loop, a bulge loop, multi-loops, a G-quadruplex, and a combination thereof.

3. The method of claim 1, wherein the first analysis data and the second analysis data each comprises a thermodynamic data for a formation of an nth-order structure in the corresponding nucleic acid sequence.

4. The method of claim 3, wherein the thermodynamic data for the formation of the nth-order structure is a thermodynamic data for a formation of an arbitrary nth-order structure, a thermodynamic data for a formation of a specific nth-order structure, or a thermodynamic data for a formation of each of a plurality of nth-order structures.

5. The method of claim 4, wherein the thermodynamic data is indicated as a change in a thermodynamic free energy.

6. The method of claim 3, wherein the thermodynamic data comprises at least one selected from the group consisting of:
a thermodynamic data for an nth-order structure present in a first block unit; wherein the first block unit is defined as a predetermined range based on a region in which an oligonucleotide is bound to the corresponding nucleic acid sequence during an annealing step of the amplification reaction;
a thermodynamic data for an nth-order structure present in a second block unit; wherein the second block is defined as a range of a region to be extended by an oligonucleotide bound to the corresponding nucleic acid sequence during an extension step of the amplification reaction; and
a thermodynamic data for an nth-order structure present in a third block unit; wherein the third block is defined as a sequence comprising (i) the second block and (ii) an additional sequence at a 5′ end and a 3′ end of the second block.

7. The method of claim 3, wherein the thermodynamic data is obtained based at least in part on the corresponding nucleic acid sequence and a reaction condition used in the amplification reaction for the corresponding nucleic acid sequence, and
wherein the reaction condition comprises a condition for a reaction medium and temperature used in the amplification reaction.

8. The method of claim 1, wherein the each training data further comprises at least one selected from the group consisting of:
(a) at least one of the nucleic acid sequence, an amplicon sequence obtained from the nucleic acid sequence, and an oligonucleotide sequence bound to the nucleic acid sequence; (b) a melting temperature (Tm) of at least one of the nucleic acid sequence, the amplicon sequence, and the oligonucleotide sequence; (c) a length of at least one of the nucleic acid sequence, the amplicon sequence, and the oligonucleotide sequence; (d) a type of the nucleic acid sequence; and (e) a GC content of at least one of the nucleic acid sequence, the amplicon sequence, and the oligonucleotide sequence.

9. The method of claim 1, wherein the amplification reaction result comprises at least one selected from the group consisting of:
(a) an amplification inhibition level representing a level at which the amplification reaction is inhibited by the nth-order structure, and (b) an amplification inhibition or an amplification non-inhibition representing whether the amplification inhibition level satisfies a predetermined criterion.

10. The method of claim 9, wherein the amplification inhibition level is calculated using a cycle value corresponding to an amplification point in a dataset comprising a signal value for each cycle for the amplification reaction.

11. The method of claim 10, wherein the cycle value corresponding to the amplification point comprises (i) a cycle value in which a primary or secondary derivative result for a curve connecting the signal value for each cycle is maximum or minimum and/or (ii) a specific cycle value in which a signal value in the dataset reaches a preset threshold value.

12. The method of claim 10, wherein the amplification inhibition level is calculated using a difference between the amplification points determined from two or more datasets obtained from two or more amplification reactions for the nucleic acid sequence.

13. The method of claim 9, wherein the predetermined criterion is determined based at least in part on a value of n and/or a type of the nth-order structure in the nucleic acid sequence, and
wherein the type of the nth-order structure comprises:
at least one selected from the group consisting of a hairpin loop, an internal loop, a bulge loop, multi-loops, G-quadruplex, and a combination thereof, when n is 2; and
at least one selected from the group consisting of a pseudoknot, a kissing hairpin, a hairpin-bulge contact, and a combination thereof, when n is 3.

14. The method of claim 1, wherein the prediction model comprises at least one selected from the group consisting of:
a machine learning-based Ridge linear regression model, a random forest regression model, a logistic regression-based classification model, and a random forest classification model.

15. The method of claim 1, wherein the prediction result comprises at least one selected from the group consisting of:
(a) an amplification inhibition level representing a level at which an amplification reaction is inhibited by the nth-order structure, (b) an amplification inhibition or an amplification non-inhibition representing whether the amplification inhibition level satisfies a predetermined criterion, and (c) a probability value for the amplification inhibition or the amplification non-inhibition.

16. The method of claim 1, wherein the input data comprises a plurality of feature, and
wherein the method further comprises, after the obtaining of the prediction result, providing a contribution level representing a level to which each of the plurality of feature contributed to the prediction result.

17. The method of claim 1, further comprising determining a designable region of an oligonucleotide based at least in part on the prediction result.

18. The method of claim 1, wherein the amplification reaction is a Polymerase chain reaction (PCR).

19. A computer device comprising:
a memory configured to store at least one instruction; and
a processor configured to execute the at least one instruction to:
access a prediction model learned using a plurality of training data, wherein each training data comprises a first analysis data for an nth-order structure in a nucleic acid sequence and an amplification reaction result for the nucleic acid sequence; wherein n is an integer not less than 2;
obtain an input data comprising a second analysis data for an nth-order structure in a target nucleic acid sequence;
provide the input data to the prediction model; and
obtain a prediction result of an amplification reaction for the target nucleic acid sequence from the prediction model.

20. A non-transitory computer-readable recording medium storing instructions which, when executed by one or more processors, cause the one or more processors to perform the method of claim 1.
